(19) United States
(12) Patent Application Publication
(10) Pub. No.: US 2009/0304631 A1
(43) Pub. Date: Dec. 10, 2009

(54) HEPATITIS C SERINE PROTEASE INHIBITORS AND USES THEREFOR

(76) Inventors: David Alan Campbell, San Diego, CA (US); Michael E. Hepperle, San Diego, CA (US); Juan Manuel Betancort, San Diego, CA (US); David T. Winn, San Diego, CA (US)

Correspondence Address: SCHWEGMAN, LUNDBERG & WOESSNER, P.A., P.O. BOX 2938, MINNEAPOLIS, MN, 55402, US

(21) Appl. No.: 12/162,268

(22) PCT Filed: Jan. 25, 2007
(86) PCT No.: PCT/US07/02225

§ 371 Date: Dec. 5, 2008

Related U.S. Application Data
(60) Provisional application No. 60/762,961, filed on Jan. 27, 2006.
(60) Provisional application No. 60/823,240, filed on Aug. 22, 2006.

(51) Int. Cl. A61K 31/69; C07F 5/02; C07D 403/12; A61K 39/395; A61K 38/16; A61K 38/21; A61P 31/12

(52) U.S. Cl. 424/85.7; 548/405; 548/465; 546/13; 514/64; 424/133.1; 514/12; 424/85.4

(57) ABSTRACT

The invention provides compounds that inhibit a viral protease enzyme of the hepatitis C virus (HCV). The compounds are adapted for treatment of a HCV infection in a patient with the disease. The compounds include analogs of tripeptides and tetrapeptides that resemble the viral protease substrate. The invention also provides pharmaceutical compositions and combinations, methods of preparation of the compounds, and methods of treatment of patients afflicted with HCV using the compounds.

### CLAIM OF PRIORITY FROM A PRIOR-FILED PROVISIONAL APPLICATION

This application claims the benefit of priority, under 35 U.S.C. Section 119(e), to U.S. Provisional Patent Applications Nos. 60/762,961, filed Jan. 27, 2006, and 60/823,240, filed Aug. 22, 2006, which are incorporated herein by reference.

### FIELD OF THE INVENTION

The present invention relates to novel compounds that are useful as protease inhibitors, particularly as inhibitors of serine proteases, and more particularly as inhibitors of the NS3 serine protease from hepatitis C virus. Because these inhibitors interfere with protease activity necessary for the hepatitis C virus, the compounds find utility as antiviral agents, especially for treatment of hepatitis C virus infections.

### BACKGROUND OF THE INVENTION

Hepatitis C virus (“HCV”) is the causative agent for hepatitis C, a chronic infection characterized by jaundice, fatigue, abdominal pain, loss of appetite, nausea, and darkening of the urine. HCV, belonging to the hepacivirus genus of the Flaviviriae family, is an enveloped, single-stranded positive-sense RNA-containing virus. The long-term effects of hepatitis C infection as a percentage of infected subjects include chronic infection (55-85%), chronic liver disease (70%), and death (1-5%). Furthermore, HCV is the leading indication for liver transplant. In chronic infection, there usually presents progressively worsening liver inflammation, which often leads to more severe disease states such as cirrhosis and hepatocellular carcinoma.

The HCV genome (Choo et al., Science 1989, 244, 359-362; Simmonds et al., Hepatology 1995, 21, 570-583) is a highly variable sequence exemplified by GenBank accession NC_{—}004102 as a 9646 base pair single-stranded RNA comprising the following constituents at the parenthetically indicated positions: 5′ NTR (i.e., non-transcribed region) (1-341); core protein (i.e., viral capsid protein involved in diverse processes including viral morphogenesis or regulation of host gene expression) (342-914); E1 protein (i.e., viral envelope) (915-1490); E2 protein (i.e., viral envelope) (1491-2579); p7 protein (2580-2768); NS2 protein (i.e., non-structural protein 2) (2769-3419); NS3 protease (3420-5312); NS4a protein (5313-5474); NS4b protein (5475-6257); NS5a protein (6258-7601); NS5b RNA-dependent RNA polymerase (7602-9372); and 3′ NTR (9375-9646). Additionally, a 17-kDalton −2/+1 frameshift protein, “protein F”, comprising the joining of positions (342-369) with (371-828) may provide functionality originally ascribed to the core protein.

The NS3 (i.e., non-structural protein 3) protein of HCV exhibits serine protease activity, the N-terminal of which is produced by the action of a NS2-NS3 metal-dependent protease, and the C-terminal of which is produced by auto-proteolysis. The HCV NS3 serine protease and its associated cofactor, NS4a, process all of the other non-structural viral proteins of HCV. Accordingly, the HCV NS3 protease is essential for viral replication.

Several compounds have been shown to inhibit the hepatitis C serine protease, but all of these have limitations in relation to the potency, stability, selectivity, toxicity, and/or pharmacodynamic properties. Such compounds have been disclosed, for example, in published U.S. patent application Nos. 2004/0266731, 2002/0032175, 2005/0137139, 2005/0119189, and 2004/9977600A1, and in published PCT patent applications WO 2005/037214 and WO 2005/035525. Accordingly, a need exists for new compounds that are useful for inhibiting the serine protease of HCV.

### SUMMARY OF THE INVENTION

The present invention provides compounds of Formula I that are adapted to inhibit the viral protease NS3 of the Hepatitis C Virus (HCV), inter alia. The compounds of Formula I are adapted to bind to, and thus block the action of, an HCV-encoded protease enzyme that is required by the virus for the production of intact, mature, functional viral proteins from the viral polyprotein as translated from the viral RNA, and therefore for the formation of infectious particles, and ultimately for viral replication. The compounds are mimics or analogs of the peptide domain immediately N-terminal of the substrate site where the viral protease cleaves its native substrate viral polyprotein.

The present invention provides a compound of Formula I:

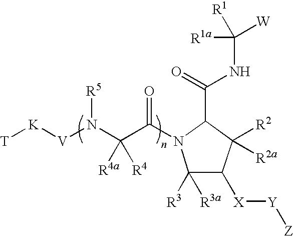

and stereoisomers, solvates, hydrates, tautomers, prodrugs, salts, pharmaceutically acceptable salts, and mixtures thereof, wherein:

n is 0 or 1;

W is

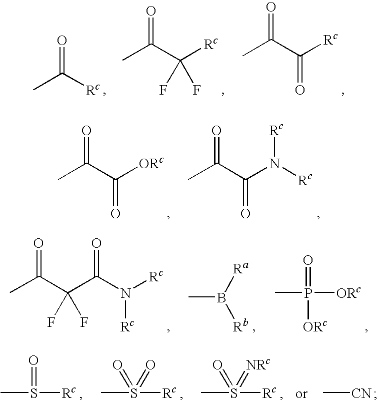

wherein

R^{a} and R^{b} are independently a hydroxyl or a group that can be converted to hydroxyl, or R^{a} and R^{b} together with the boron to which they are attached form a cyclic group which can be converted to a —B(OH)₂ group;

R^{c} at each occurrence is independently H, substituted or unsubstituted alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroarylalkyl; or two R^{c} groups bound to a nitrogen atom can together with the nitrogen atom to which they are bound form a 5-7 membered monocyclic heterocyclic ring system; wherein any carbon atom of R^{c} can be substituted with J;

R¹ and R^{1a} are independently H or a substituted or unsubstituted alkyl, alkenyl, alkynyl, cycloalkyl, cycloalkylalkyl, aryl, or aralkyl group; or R¹ and R^{1a} together with a carbon atom to which they are attached form together with the carbon atom a 3-7 membered substituted or unsubstituted carbocycle; wherein each of R¹ or R^{1a} can be substituted with 0-3 J;

R², R^{2a}, R³ and R^{3a} are independently H or a substituted or unsubstituted alkyl, alkenyl, aryl, aralkyl, aralkenyl, cycloalkyl, cycloalkylalkyl, cycloalkenyl, cycloalkenylalkyl, heterocyclyl, heterocyclylalkyl, heteroaryl, or heteroarylalkyl group, wherein any carbon atom can be substituted with J;

R⁴ and R^{4a} are independently hydrogen, (C₁-C₁₂)-aliphatic, (C₃-C₁₀)-cycloalkyl or cycloalkenyl, [(C₃-C₁₀)cycloalkyl or cycloalkenyl]-(C₁-C₁₂)-aliphatic, (C₆-C₁₀)-aryl, (C₆-C₁₀)-aryl-(C₁-C₁₂)-aliphatic, (C₃-C₁₀)-heterocyclyl, (C₃-C₁₀)-heterocyclyl-(C₁-C₁₂)-aliphatic, (C5-C₁₀)-heteroaryl, or (C₅-C₁₀)-heteroaryl-(C₁-C₁₂)-aliphatic;
wherein each (C₁-C₁₂)-aliphatic, (C₃-C₁₀)-cycloalkyl or cycloalkenyl, [(C₃-C₁₀)cycloalkyl or cycloalkenyl]-(C₁-C₁₂)-aliphatic, (C₆-C₁₀)-aryl, (C₆-C₁₀)-aryl-(C₁-C₁₂)-aliphatic, (C₃-C₁₀)-heterocyclyl, (C₃-C₁₀)-heterocyclyl-(C₁-C₁₂)-aliphatic, (C₅-C₁₀)-heteroaryl, or (C₅-C₁₀)-heteroaryl-(C₁-C₁₂)-aliphatic of R⁴ or R^{4a} is independently substituted with 0-3 substituents independently selected from J;
wherein up to 3 carbon atoms in each of R⁴ or R^{4a} may be replaced by a heteroatom selected from N, NH, O, S, SO, or SO₂ in a chemically stable arrangement; or wherein R⁴ and R^{4a} together with a carbon atom to which they are bound form a 3- to 8-membered ring having up to 3 heteroatoms selected from N, NH, O, S, SO, or SO₂, wherein the ring system is substituted with 0-2 substituents selected independently from J;

R⁵ is hydrogen, alkyl, alkenyl, cycloalkyl, cycloalkylalkyl, cycloalkenyl, cycloalkyenylalkyl, aryl, aralkyl, heterocyclyl, heterocyclylalkyl, heteroaryl or heteroarylalkyl; wherein the alkyl, alkenyl, cycloalkyl, cycloalkylalkyl, cycloalkenyl, cycloalkenylalkyl, aryl, aralkyl, heterocyclyl, heterocyclylalkyl, heteroaryl or heteroarylalkyl is substituted with 0-3 J groups;

X is a bond, C(H)R⁷, O, S, or N(R⁷);

Y is a bond, C(H)R⁷, C(O), C(O)C(O), S(O), S(O)₂, or S(O)(NR⁷);
wherein R⁷ is hydrogen, alkyl, cycloalkyl, cycloalkylalkyl, aryl, aralkyl, heterocyclyl, heterocyclylalkyl, heteroaryl, heteroarylalkyl, aralkanoyl, heteroaralkanoyl, C(O)R⁸, C(O)OR⁸, SO₂R⁸, or carboxamido, and the alkyl, cycloalkyl, cycloalkylalkyl, aryl, aralkyl, heterocyclyl, heterocyclylalkyl, heteroaryl, heteroarylalkyl, aralkanoyl, or heteroaralkanoyl is substituted with 0-3 J groups, or R⁷ and Z, together with the atoms to which they are bound, form a mono- or bicyclic ring system substituted with 0-3 J groups;
wherein R⁸ is alkyl, aryl, aralkyl, heterocyclyl, heterocyclylalkyl, heteroaryl or heteroarylalkyl, any of which is substituted with 0-3 J groups;

Z is alkyl, aryl, aralkyl, cycloalkyl, cycloalkylalkyl, cycloalkenyl, cycloalkenylalkyl, heterocyclyl, heterocyclylalkyl, heteroaryl, heteroarylalkyl, OR⁹, or N(R⁹)₂, wherein any carbon atom can be substituted with J;
wherein each R⁹ is independently hydrogen, alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroarylalkyl, the alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroarylalkyl being substituted with 0-3 J groups; or two R⁹ groups which are bound to a nitrogen atom form together with the nitrogen atom a 3- to 8-membered mono-, or an 8- to 20-membered bi- or tricyclic heterocyclic ring system substituted with 0-3 J groups;

J is halogen, OR′, OC(O)N(R′)₂, NO₂, CN, CF₃, OCF₃, R′, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, (CH₂)₀₋₂N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, C(═NOR′)R′, OP(O)(OR′)₂, P(O)(R′)₂, P(O)(OR)₂, or P(O)(H)(OR′); or two J groups taken together are O, S, C(O), S(O), S(O)₂, methylenedioxy, or ethylenedioxy;
wherein each R′ is independently selected from hydrogen, (C₁-C₁₂)-aliphatic, (C₃-C₃-C₁₀)-cycloalkyl or cycloalkenyl, [(C₃-C₁₀)cycloalkyl or cycloalkenyl]-(C₁-C₁₂)-aliphatic, (C₆-C₁₀)-aryl, (C₆-C₁₀)-aryl-(C₁-C₁₂)-aliphatic, (C₃-C₁₀)-heterocyclyl, (C₃-C₁₀)-heterocyclyl-(C₁-C₁₂)-aliphatic, (C₅-C₁₀)-heteroaryl, or (C₅-C₁₀)-heteroaryl-(C₁-C₁₂)-aliphatic, or wherein two R′ groups together with a nitrogen atom to which they are bound form together with the nitrogen atom a 3- to 8-membered mono-, or an 8- to 20-membered bi- or tricyclic heterocyclic ring system; wherein, in the bi- and tricyclic ringsystems, each ring is linearly fused, bridged, or spirocyclic; wherein each ring is either aromatic or nonaromatic; wherein each heteroatom in the heterocyclic ring system is selected from the group consisting of N, NH, O, S, SO and SO₂,
wherein R′ other than hydrogen is substituted with 0-3 substituents selected independently from J;

V is a bond, CH₂, C(R¹⁰)₂, C(O), S(O), or S(O)₂;

K is a bond, —O—, —S—, —C(O)—, —S(O)—, S(O)₂—, or —S(O)(NR¹⁰)—, —N(R¹⁰)—;
wherein R¹⁰ is hydrogen or C₁₋₅ alkyl;

T is R¹¹, R¹¹-alkyl-, R¹¹-alkenyl-, R¹¹-alkynyl-, R¹¹O—, —N(R¹¹)₂, —C(O) R¹¹, or

### —C(═NOalkyl) R¹¹;

wherein R¹¹ is independently hydrogen, alkyl, aryl, aralkyl, alkoxy, cycloalkyl, cycloalkylalkyl, cycloalkenyl, cycloalkenylalkyl, heterocyclyl, heterocyclylalkyl, heterocyclylalkenyl, or heteroaryl, and each alkyl, aryl, aralkyl, alkoxy, cycloalkyl, cycloalkylalkyl, cycloalkenyl, cycloalkenylalkyl, heterocyclyl, heterocyclylalkyl, heterocyclylalkenyl, or heteroaryl is substituted with 0-3 J groups; or a first R^{1l} and a second R¹¹ bonded to a nitrogen atom together with the nitrogen atom to which they are bound form a mono- or bicyclic ring system substituted with 0-3 J groups.

The invention further provides a pharmaceutical composition comprising a compound of Formula I and a suitable excipient.

The invention further provides a pharmaceutical combination comprising a compound of Formula I in a therapeutically effective amount and a second medicament in a therapeutically effective amount. The invention also provides a pharmaceutical combination comprising a compound of Formula I in a therapeutically effective amount, a second medicament in a therapeutically effective amount, and a third medicament in a therapeutically effective amount. The pharmaceutical combinations of the invention may be formulated as pharmaceutical compositions of the invention.

The present invention further provides a method of treatment of a HCV infection in a patient in need thereof, or in a patient when inhibition of an HCV viral protease is medically indicated, comprising administering a therapeutically effective amount of a compound of Formula I to the patient, or a pharmaceutical combination to the patient.

The present invention further provides a method of use of a compound of Formula I in preparation of a medicament for the treatment of Hepatitis C.

### DETAILED DESCRIPTION

### Definitions

The terms “HCV NS3 serine protease”, “HCV NS3 protease”, “NS3 serine protease”, and “NS3 protease” denote all active forms of the serine protease encoded by the NS3 region of the hepatitis C virus, including all combinations thereof with other proteins in either covalent or noncovalent association. For example, other proteins in this context include without limitation the protein encoded by the NS4a region of the hepatitis C virus. Accordingly, the terms “NS3/4a” and “NS3/4a protease” denote the NS3 protease in combination with the HCV NS4a protein.

The term “other type(s) of therapeutic agents” as employed herein refers to one or more antiviral agents (other than HCV NS3 serine protease inhibitors of the invention).

“Subject” as used herein, includes mammals such as humans, non-human primates, rats, mice, dogs, cats, horses, cows and pigs.

The term “treatment” is defined as the management and care of a patient for the purpose of combating the disease, condition, or disorder and includes administering a compound of the present invention to prevent the onset of the symptoms or complications, or alleviating the symptoms or complications, or eliminating the disease, condition, or disorder.

“Treating” within the context of the instant invention means an alleviation of symptoms associated with a disorder or disease, or inhibition of further progression or worsening of those symptoms, or prevention or prophylaxis of the disease or disorder. Thus, treating a hepatitis C viral infection includes slowing, halting or reversing the growth of the virus and/or the control, alleviation or prevention of symptoms of the infection. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result by inhibition of HCV NS3 serine protease activity. A therapeutically effective amount is also one in which any toxic or detrimental effects of compounds of the invention are outweighed by the therapeutically beneficial effects. For example, in the context of treating HCV infection, a therapeutically effective amount of a HCV NS3 serine protease inhibitor of the invention is an amount sufficient to control HCV viral infection.

All chiral, diastereomeric, racemic forms of a structure are intended, unless the specific stereochemistry or isomeric form is specifically indicated. Compounds used in the present invention include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these are all within the scope of the invention.

The term “amino protecting group” or “N-protected” as used herein refers to those groups intended to protect an amino group against undesirable reactions during synthetic procedures and which can later be removed to reveal the amine. Commonly used amino protecting groups are disclosed in Protective Groups in Organic Synthesis, Greene, T. W.; Wuts, P. G. M., John Wiley & Sons, New York, N.Y., (3rd Edition, 1999). Amino protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups (which form urethanes with the protected amine) such as benzyloxycarbonyl(Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl(Alloc), 2,2,2-trichloroethoxycarbonyl, 2-trimethylsilylethyloxycarbonyl(Teoc), phenoxycarbonyl, 4-nitrophenoxycarbonyl, fluorenyl-9-methoxycarbonyl(Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Amine protecting groups also include cyclic amino protecting groups such as phthaloyl and dithiosuccinimidyl, which incorporate the amino nitrogen into a heterocycle. Typically, amino protecting groups include formyl, acetyl, benzoyl, pivaloyl, t-butylacetyl, phenylsulfonyl, Alloc, Teoc, benzyl, Fmoc, Boc and Cbz. It is well within the skill of the ordinary artisan to select and use the appropriate amino protecting group for the synthetic task at hand.

Alkyl groups include straight chain and branched alkyl groups and cycloalkyl groups having from 1 to about 20 carbon atoms, and typically from 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. Representative substituted alkyl groups may be substituted one or more times with any of the groups listed below, for example, amino, hydroxy, cyano, carboxy, nitro, thio, alkoxy, and halogen groups.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which may be substituted with, for example, amino, hydroxy, cyano, carboxy, nitro, thio, alkoxy, and halogen groups. The term “cycloalkenyl” alone or in combination denotes a cyclic alkenyl group.

The terms “carbocyclic” and “carbocycle” denote a ring structure wherein the atoms of the ring are carbon. In some embodiments, the carbocycle has 3 to 8 ring members, whereas in other embodiments the number of ring carbon atoms is 4, 5, 6, or 7. Unless specifically indicated to the contrary, the carbocyclic ring may be substituted with as many as N-1 substituents wherein N is the size of the carbocyclic ring with for example, amino, hydroxy, cyano, carboxy, nitro, thio, alkoxy, and halogen groups.

(Cycloalkyl)alkyl groups, also denoted cycloalkylalkyl, are alkyl groups as defined above in which a hydrogen or carbon bond of the alkyl group is replaced with a bond to a cycloalkyl group as defined above.

Alkenyl groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

Cycloalkenyl groups include cycloalkyl groups having at least one double bond between 2 carbons. Thus for example, cycloalkenyl groups include but are not limited to cyclohexenyl, cyclopentenyl, cyclohexadienyl, butadienyl, pentadienyl, and hexadienyl groups.

(Cycloalkenyl)alkyl groups are alkyl groups as defined above in which a hydrogen or carbon bond of the alkyl group is replaced with a bond to a cycloalkenyl group as defined above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

Aryl groups are cyclic aromatic hydrocarbons that do not contain heteroatoms. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain 6-14 carbons in the ring portions of the groups. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halogen groups, bonded to one of the ring members. Rather, groups such as tolyl are referred to as substituted aryl groups. Representative substituted aryl groups may be mono-substituted or substituted more than once, such as, but not limited to, 2-, 3-, 4-, 5-, or 6-substituted phenyl or naphthyl groups, which may be substituted with groups such as those listed below.

Aralkyl groups are alkyl groups as defined above in which a hydrogen or carbon bond of an alkyl group is replaced with a bond to an aryl group as defined above. Representative aralkyl groups include benzyl and phenylethyl groups and fused (cycloalkylaryl)alkyl groups such as 4-ethyl-indanyl. Aralkenyl group are alkenyl groups as defined above in which a hydrogen or carbon bond of an alkyl group is replaced with a bond to an aryl group as defined above.

Heterocyclyl groups include aromatic and non-aromatic ring compounds containing 3 or more ring members, of which, one or more is a heteroatom such as, but not limited to, N, O, and S. In some embodiments, heterocyclyl groups include 3 to 20 ring members, whereas other such groups have 3 to 15 ring members. The phrase “heterocyclyl group” includes fused ring species including those comprising fused aromatic and non-aromatic groups. The phrase also includes polycyclic ring systems containing a heteroatom such as, but not limited to, quinuclidyl. However, the phrase does not include heterocyclyl groups that have other groups, such as alkyl or halogen groups, bonded to one of the ring members. Rather, these are referred to as “substituted heterocyclyl groups”. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofaranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups.

Representative substituted heterocyclyl groups may be mono-substituted or substituted more than once, such as, but not limited to, piperidinyl or quinolinyl groups, which are 2-, 3-, 4-, 5-, or 6-substituted, or disubstituted with groups such as those listed below.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Although the phrase “heteroaryl groups” includes fused ring compounds such as indolyl and 2,3-dihydro indolyl, the phrase does not include heteroaryl groups that have other groups bonded to one of the ring members, such as alkyl groups. Rather, heteroaryl groups with such substitution are referred to as “substituted heteroaryl groups”. Representative substituted heteroaryl groups may be substituted one or more times with groups such as those listed above.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine(10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

Heterocyclylalkyl groups are alkyl groups as defined above in which a hydrogen or carbon bond of an alkyl group is replaced with a bond to a heterocyclyl group as defined above. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

Heteroaralkyl groups are alkyl groups as defined above in which a hydrogen or carbon bond of an alkyl group is replaced with a bond to a heteroaryl group as defined above.

The term “alkoxy” refers to an oxygen atom connected to an alkyl group as defined above. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like.

The terms “aryloxy” and “arylalkoxy” refer to, respectively, an aryl group bonded to an oxygen atom and an aralkyl group bonded to the oxygen atom at the alkyl. Examples include but are not limited to phenoxy, naphthyloxy, and benzyloxy.

The term “amine” (or “amino”) includes primary, secondary, and tertiary amines having, e.g., the formula —NR³⁰R³¹. R³⁰ and R³¹ at each occurrence are independently hydrogen, or a substituted or unsubstituted alkyl, alkenyl, alkynyl, cycloalkyl, cycloalkenyl, aryl, aralkyl, heterocyclyl or heterocyclylalkyl group as defined herein. Amines thus include but are not limited to —NH₂, alkylamines, dialkylamines, arylamines, alkylarylamines, diarylamines, aralkylamines, heterocyclylamines and the like.

The term “amide” (or “amido”) includes C- and N-amide groups, i.e., —C(O)NR³²R³³, and —NR³²C(O)R³³ groups, respectively. R³² and R³³ are independently hydrogen, or a substituted or unsubstituted alkyl, alkenyl, alkynyl, cycloalkyl, cycloalkenyl, aryl, aralkyl, heterocyclyl or heterocyclylalkyl group as defined herein. Amide groups therefore include but are not limited to carbamoyl groups (—C(O)NH₂) and formamide groups (—NHC(O)H).

The term “halo” or “halogen” refer to fluorine, chlorine, bromine, or iodine.

Functional groups such as “cyano,” “nitro,” “trifluoromethyl,” “trifluoromethoxy,” and the like have the usual meaning in the art.

The term “urethane” (or “carbamyl”) includes N- and O-urethane groups, i.e., —NR³⁴C(O)OR³⁵ and —OC(O)NR³⁴R³⁵ groups, respectively. R³⁴ and R³⁵ are independently hydrogen, or a substituted or unsubstituted alkyl, cycloalkyl, aryl, aralkyl, or heterocyclyl group as defined herein.

The term “sulfonamide” (or “sulfonamido”) includes S- and N-sulfonamide groups, i.e., —SO₂NR³⁶R³⁷ and —NR³⁶SO₂R³⁷ groups, respectively. R³⁶ and R³⁷ are independently hydrogen, or a substituted or unsubstituted alkyl, cycloalkyl, aryl, aralkyl, or heterocyclyl group as defined herein. Sulfonamide groups therefore include but are not limited to sulfamoyl groups (—SO₂NH₂). An organosulfur structure represented by the formula —S(O)(NR)— is understood to refer to a sulfoximine, wherein both the oxygen and the nitrogen atoms are bonded to the sulfur atom, which is also bonded to two carbon atoms.

The term “amidine” or “amidino” includes groups of the formula —C(NR³⁸)NR³⁹R⁴⁰. R³⁸, R³⁹, and R⁴⁰ are independently H, an amino protecting group, or a substituted or unsubstituted alkyl, cycloalkyl, aryl, aralkyl, or heterocyclyl group as defined herein. Typically, an amidino group is —C(NH)NH₂.

The term “guanidine” or “guanidino” includes groups of the formula —NR⁴¹C(NR⁴²)NR⁴³R⁴⁴. R⁴¹, R⁴², R⁴³, and R⁴⁴ are independently H, an amino protecting group, or a substituted or unsubstituted alkyl, cycloalkyl, aryl, aralkyl, or heterocyclyl group as defined herein. Typically, a guanidino group is —NHC(NH)NH₂.

The term “alkylene” denotes a divalent alkyl. Examples of alkylene include, without limitation, methylene, ethylene, propylene, and the like. The term “carboxyalkyl” includes groups of the formula —R⁴⁵—COOH wherein R⁴⁵ is a substituted or unsubstituted alkylene. The term “carboxamidoalkyl” includes groups of the formula —R⁴⁵—OC(O)NR⁴³R⁴⁴ wherein R⁴⁵, R⁴³ and R⁴⁴ are as defined above. The term “heteroarylalkyl” includes groups of formula —R⁴⁵-heteroaryl, wherein R⁴⁵ and heteroaryl are as defined above. The term cycloalkylidenyl, alone or in combination with any other term, refers to a stable carbocyclic ring radical containing at least one exocyclic carbon-carbon double bond. Preferably, a cycloalkylidenyl has from 5-7 carbon atoms. Examples of cycloalkylidenyl include, without limitation, cyclopentylidenyl, cyclohexylidenyl, cyclopentenylidenyl, and the like. The term heterocycloalkylidenyl, alone or in combination with any other term, refers to a stable heterocyclic ring radical containing at least one exocyclic carbon-carbon double bond.

In general, “substituted” refers to an organic group as defined (e.g., alkyl, aryl, cycloalkyl, aralkyl, heterocyclyl, heteroaryl, etc.) in which one or more bonds to a hydrogen atom contained therein is replaced by a bond to a non-hydrogen atom such as, but not limited to: a halogen (F, Cl, Br, and I); an oxygen atom in groups such as hydroxyl groups that can be free or can be blocked as with a hydroxyl protecting group such as a silyl ether, in ethers such as alkoxy or aryloxy groups, aryloxy groups, and aralkyloxy groups, in acyloxy groups such as carboxy esters, carbamyl esters, carbonate esters and the like, and in inorganic esters such as boronate, phosphate, phosphonate, phosphinate, sulfenate, sulfinate, or sulfonate esters; a carbon atom in groups such as cyano, carboxyl, acyl, ester, amide and the like; a sulfur atom in groups such as thiol groups, alkyl and aryl sulfide groups, sulfoxide groups, sulfone groups, alkoxy- or aryloxy-sulfonyl groups, and sulfonamide groups; a nitrogen atom in groups such as nitro, amines, hydroxylamines, N-oxides, hydrazides, azides, and enamines; and other covalently bonded heteroatoms, such as phosphorus in groups such as phosphonates and phosphinates. The organic group as defined can also be substituted with groups wherein more than one bond to hydrogen atoms on a carbon atom are replaced by two or more distinct bonds to two or three heteroatoms atoms of a single substituent group, or alternatively including double or triple bonds to a heteroatom such as, but not limited to: oxygen in carbonyl(oxo), two oxygens as in cyclic acetals, hemiacetals, ketals, and hemiketals; three oxygens as in ortho-esters, an oxygen and a nitrogen as in cyclic aminals and hemiaminals; nitrogen as in imines, hydroxyimines, oximes, hydrazones, and nitrites; sulfur such as in thiocarbonyls; and phosphorus as in phosphorus ylidene compounds.

The term “heteroatoms” as used herein refers to non-carbon and non-hydrogen atoms, and is not otherwise limited. Typical heteroatoms are N, O, and S. When sulfur (S) is referred to, it is understood that the sulfur can be in any of the oxidation states in which it is found, thus including sulfoxides (R—S(O)—R′) and sulfones (R′—S(O)₂—R′), unless the oxidation state is specified; thus, the term “sulfone” encompasses only the sulfone form of sulfur; the term “sulfide” encompasses only the sulfide (R′—S—R′) form of sulfur. When the phrases such as “heteroatoms selected from the group consisting of O, NH, NR′ and S,” or “[variable] is O, S′ . . . ” are used, they are understood to encompass all of the sulfide, sulfoxide and sulfone oxidation states of sulfur, wherein sulfur is also bonded to two carbon atoms.

Substituted ring groups such as substituted aryl, heterocyclyl and heteroaryl groups also include rings and fused ring systems in which a bond to a hydrogen atom is replaced with a bond to a carbon atom. Therefore, substituted aryl, heterocyclyl and heteroaryl groups may also be substituted with alkyl, alkenyl, and alkynyl groups as defined herein.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

### Compounds of Formula I

The present invention provides a compound of Formula I:

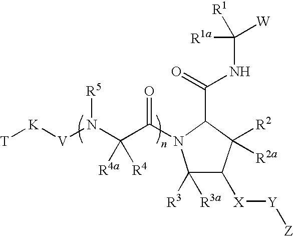

and stereoisomers, solvates, hydrates, tautomers, prodrugs, salts, pharmaceutically acceptable salts, and mixtures thereof, wherein:

n is 0 or 1;

W is

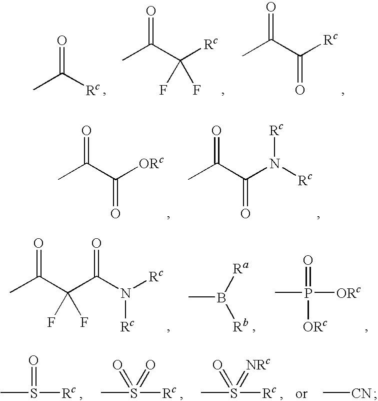

wherein

R^{a} and R^{b} are independently a hydroxyl or a group that can be converted to hydroxyl, or R^{a} and R^{b} together with the boron to which they are attached form a cyclic group which can be converted to a —B(OH)₂ group;

R^{c} at each occurrence is independently H, substituted or unsubstituted alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroarylalkyl; or two R^{c} groups bound to a nitrogen atom can together with the nitrogen atom to which they are bound form a 5-7 membered monocyclic heterocyclic ring system; wherein any carbon atom of R^{c} can be substituted with J;

R¹ and R^{1a} are independently H or a substituted or unsubstituted alkyl, alkenyl, alkynyl, cycloalkyl, cycloalkylalkyl, aryl, or aralkyl group; or R¹ and R^{1a} together with a carbon atom to which they are attached form together with the carbon atom a 3-7 membered substituted or unsubstituted carbocycle; wherein each of R¹ or R^{1a} can be substituted with 0-3 J;

R², R^{2a}, R³ and R^{3a} are independently H or a substituted or unsubstituted alkyl, alkenyl, aryl, aralkyl, aralkenyl, cycloalkyl, cycloalkylalkyl, cycloalkenyl, cycloalkenylalkyl, heterocyclyl, heterocyclylalkyl, heteroaryl, or heteroarylalkyl group, wherein any carbon atom can be substituted with J;

R⁴ and R^{4a} are independently hydrogen, (C₁-C₁₂)-aliphatic, (C₃-C₁₀)-cycloalkyl or cycloalkenyl, [(C₃-C₁₀)cycloalkyl or cycloalkenyl]-(C₁-C₁₂)-aliphatic, (C₆-C₁₀)-aryl, (C₆-C₁₀)-aryl-(C₁-C₂)-aliphatic, (C₃-C₁₀)-heterocyclyl, (C₃-C₁₀)-heterocyclyl-(C₁-C₁₂)-aliphatic, (C₅-C₁₀)-heteroaryl, or (C₅-C₁₀)-heteroaryl-(C₁-C₁₂)-aliphatic;
wherein each (C₁-C₁₂)-aliphatic, (C₃-C₁₀)-cycloalkyl or cycloalkenyl, [(C₃-C₁₀)cycloalkyl or cycloalkenyl]-(C₁-C₁₂)-aliphatic, (C₆-C₁₀)-aryl, (C₆-C₁₀)-aryl-(C₁-C₁₂)-aliphatic, (C₃-C₁₀)-heterocyclyl, (C₃-C₁₀)-heterocyclyl-(C₁-C₁₂)-aliphatic, (C₅-C₁₀)-heteroaryl, or (C₅-C₁₀)-heteroaryl-(C₁-C₁₂)-aliphatic of R⁴ or R^{4a} is independently substituted with 0-3 substituents independently selected from J;
wherein up to 3 carbon atoms in each of R⁴ or R^{4a} may be replaced by a heteroatom selected from N, NH, O, S, SO, or SO₂ in a chemically stable arrangement; or wherein R⁴ and R^{4a} together with a carbon atom to which they are bound form a 3- to 8-membered ring having up to 3 heteroatoms selected from N, NH, O, S, SO, or SO₂, wherein the ring system is substituted with 0-2 substituents selected independently from J;

R⁵ is hydrogen, alkyl, alkenyl, cycloalkyl, cycloalkylalkyl, cycloalkenyl, cycloalkyenylalkyl, aryl, aralkyl, heterocyclyl, heterocyclylalkyl, heteroaryl or heteroarylalkyl; wherein the alkyl, alkenyl, cycloalkyl, cycloalkylalkyl, cycloalkenyl, cycloalkenylalkyl, aryl, aralkyl, heterocyclyl, heterocyclylalkyl, heteroaryl or heteroarylalkyl is substituted with 0-3 J groups;

X is a bond, C(H)R⁷, O, S, or N(R⁷);

Y is a bond, C(H)R⁷, C(O), C(O)C(O), S(O), S(O)₂, or S(O)(NR⁷);
wherein R⁷ is hydrogen, alkyl, cycloalkyl, cycloalkylalkyl, aryl, aralkyl, heterocyclyl, heterocyclylalkyl, heteroaryl, heteroarylalkyl, aralkanoyl, heteroaralkanoyl, C(O)R⁸, C(O)OR⁸, SO₂R⁸, or carboxamido, and the alkyl, cycloalkyl, cycloalkylalkyl, aryl, aralkyl, heterocyclyl, heterocyclylalkyl, heteroaryl, heteroarylalkyl, aralkanoyl, or heteroaralkanoyl is substituted with 0-3 J groups, or R⁷ and Z, together with the atoms to which they are bound, form a mono- or bicyclic ring system substituted with 0-3 J groups;
wherein R⁸ is alkyl, aryl, aralkyl, heterocyclyl, heterocyclylalkyl, heteroaryl or heteroarylalkyl, any of which is substituted with 0-3 J groups;

Z is alkyl, aryl, aralkyl, cycloalkyl, cycloalkylalkyl, cycloalkenyl, cycloalkenylalkyl, heterocyclyl, heterocyclylalkyl, heteroaryl, heteroarylalkyl, OR⁹, or N(R⁹)₂, wherein any carbon atom can be substituted with J;
wherein each R⁹ is independently hydrogen, alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroarylalkyl, the alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroarylalkyl being substituted with 0-3 J groups; or two R⁹ groups which are bound to a nitrogen atom form together with the nitrogen atom a 3- to 8-membered mono-, or an 8- to 20-membered bi- or tricyclic heterocyclic ring system substituted with 0-3 J groups;

J is halogen, OR′, OC(O)N(R′)₂, NO₂, CN, CF₃, OCF₃, R′, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, (CH₂)₀₋₂N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, C(═NOR′)R′, OP(O)(OR′)₂, P(O)(R′)₂, P(O)(OR′)₂, or P(O)(H)(OR′); or two J groups taken together are O, S, C(O), S(O), S(O)₂, methylenedioxy, or ethylenedioxy;
wherein each R′ is independently selected from hydrogen, (C₁-C₁₂)-aliphatic, (C₃-C₁₀)-cycloalkyl or cycloalkenyl, [(C₃-C₁₀)cycloalkyl or cycloalkenyl]-(C₁-C₁₂)-aliphatic, (C₆-C₁₀)-aryl, (C₆-C₁₀)-aryl-(C₁-C₁₂)-aliphatic, (C₃-C₁₀)-heterocyclyl, (C₃-C₁₀)-heterocyclyl-(C₁-C₁₂)-aliphatic, (C₅-C₁₀)-heteroaryl, or (C₅-C₁₀)-heteroaryl-(C₁-C₁₂)-aliphatic, or wherein two R′ groups together with a nitrogen atom to which they are bound form together with the nitrogen atom a 3- to 8-membered mono-, or an 8- to 20-membered bi- or tricyclic heterocyclic ring system; wherein, in the bi- and tricyclic ringsystems, each ring is linearly fused, bridged, or spirocyclic; wherein each ring is either aromatic or nonaromatic; wherein each heteroatom in the heterocyclic ring system is selected from the group consisting of N, NH, O, S, SO and SO₂,
wherein R′ other than hydrogen is substituted with 0-3 substituents selected independently from J;

V is a bond, CH₂, C(R¹⁰)₂, C(O), S(O), or S(O)₂;

K is a bond, —O—, —S—, —C(O)—, —S(O)—, S(O)₂—, or —S(O)(NR¹⁰)—, —N(R¹⁰)—;
wherein R¹⁰ is hydrogen or C₁₋₅ alkyl;

T is R¹¹, R¹¹-alkyl-, R¹¹-alkenyl-, R¹¹-alkynyl-, R¹¹O—, —N(R¹¹)₂, —C(O) R¹¹, or

### —C(═NOalkyl) R¹¹;

wherein R¹¹ is independently hydrogen, alkyl, aryl, aralkyl, alkoxy, cycloalkyl, cycloalkylalkyl, cycloalkenyl, cycloalkenylalkyl, heterocyclyl, heterocyclylalkyl, heterocyclylalkenyl, or heteroaryl, and each alkyl, aryl, aralkyl, alkoxy, cycloalkyl, cycloalkylalkyl, cycloalkenyl, cycloalkenylalkyl, heterocyclyl, heterocyclylalkyl, heterocyclylalkenyl, or heteroaryl is substituted with 0-3 J groups; or a first R¹¹ and a second R¹¹ bonded to a nitrogen atom together with the nitrogen atom to which they are bound form a mono- or bicyclic ring system substituted with 0-3 J groups.

More specifically, Z can be:

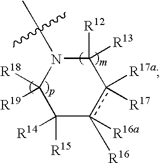

wherein

the bond including a dashed line can be a single bond or a double bond;

m is 0 or 1;

p is 0 or 1;

R¹², R¹³, R¹⁸, and R¹⁹ are independently hydrogen or a substituted or unsubstituted alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group; or R¹² and R¹³, or R¹⁸ and R¹⁹, together with a carbon atom to which they are attached, form a C₃₋₆ cycloalkyl group;

R¹⁴ and R¹⁵ are independently hydrogen, fluorine, or a substituted or unsubstituted alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group, or R¹⁴ and R¹⁵, together with a carbon atom to which they are attached, form a C₃₋₆ cycloalkyl group;
wherein any R¹², R¹³, R¹⁴, R¹⁵, R¹⁸, or R¹⁹ alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group is substituted with 0-3 J groups; and wherein any C₃₋₆ cycloalkyl group formed by R¹² and R¹³, or R¹⁴ and R¹⁵, or R¹⁸ and R¹⁹, together with a carbon atom to which they are bonded, may comprise 1 or 2 heteroatoms selected from a group consisting of O, NH, NR′, S, SO, and SO₂;

R¹⁶, R^{16a}, R¹⁷ and R^{17a} are independently hydrogen or a substituted or unsubstituted alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group; or R¹⁶ and R¹⁷ together with the atoms to which they are attached form a fused substituted or unsubstituted aryl or heteroaryl group; or when the bond including the dashed line is a double line, R^{16a} and R^{17a} are absent. For example, R¹², R¹³, R¹⁴, R¹⁵, R¹⁶, R^{16a}, R¹⁷, R^{17a}, R¹⁸ and R¹⁹ can be hydrogen. When the bond including a dashed line is a double bond, R^{16a} and R^{17a} are absent.

Alternatively, Z can be:

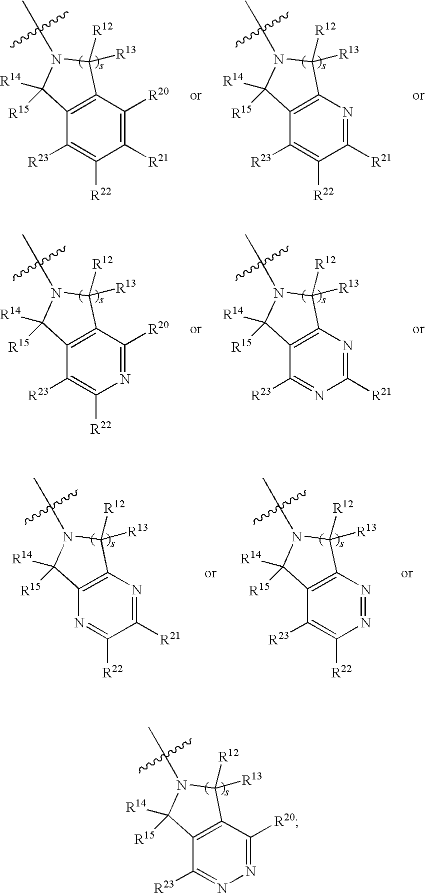

wherein s is 1 or 2;

R¹², R¹³, R¹⁴, and R¹⁵ are at each occurrence independently hydrogen, fluorine, or an alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group; or R¹² and R¹³, or R¹⁴ and R¹⁵, together with a carbon atom to which they are bonded, form a C₃₋₆ cycloalkyl group;
wherein any R¹², R¹³, R¹⁴, or R¹⁵ alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group is substituted with 0-3 J groups; and wherein any C₃₋₆ cycloalkyl group formed by R¹² and R¹³, or R¹⁴ and R¹⁵, together with a carbon atom to which they are bonded, may comprise 1 or 2 heteroatoms selected from a group consisting of O, NH, NR′, S, SO, and SO₂;

R²⁰, R²¹, R²², R²³ are a substituted or unsubstituted alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group, or are independently H, F, Cl, Br, I, NO₂, CN, CF₃, OR²⁴, O—(CH₂)ᵣ—NR²⁵R²⁶, O—(CH₂)ᵣ—OC(O)NR²⁵R²⁶, O—(CH₂)ᵣ—NR²⁵C(O)OR²⁶, (CH₂)ᵣ—OR²⁴, OCF₃, NR²⁵R²⁶, (CH₂)ᵣ—NR²⁵R²⁶, SR²⁴, (CH₂)ᵣ—SR²⁴, C(O)R²⁴, C(O)OR²⁴, NR²⁷C(O)R²⁴, C(O)NR²⁵R²⁶, NR²⁷C(O)NR²⁵R²⁶, OC(O)NR²⁵R²⁶, NR²⁷C(O)OR²⁴, NR²⁷SO R²⁴, SO₂NR²⁵R²⁶, wherein r is 1, 2, 3, 4, 5, or 6; and

each R²⁴, R²⁵, R²⁶, and R²⁷ is independently hydrogen or an alkyl, cycloalkyl, cycloalkenyl, cycloalkylalkyl, cycloalkenylalkyl, cycloalkylalkenyl, aryl, aralkyl, arylalkenyl, heterocyclyl, heterocyclylalkyl, heterocyclylalkenyl, heteroaryl, heteroarylalkyl, or heteroarylalkenyl group, wherein any group except hydrogen is substituted with 0-3 J groups; or R²⁵ and R²⁶ together with a nitrogen atom to which they are attached form together with the nitrogen atom a 3-7 membered heterocyclic ring which is substituted with 0-3 J groups. For example, R¹², R¹³, R¹⁴, R¹⁵, R²⁰, R²¹, R²², and R²³ can be hydrogen.

In another embodiment, R¹ is alkyl, cycloalkyl, or cycloalkylalkyl, and R^{1a} is H; or R¹ and R^{1a} together with a carbon atom to which they are attached form together with the carbon atom a 3-, 4-, or 5-membered cycloalkyl, wherein any 1 or 2 carbon atoms of R¹, or of R¹ and R^{1a} combined in the cycloalkyl, may be replaced by a heteroatom selected from the group consisting of O, NH, NR′, S, SO or SO₂; wherein any carbon atom of R¹ or of R¹ and R^{1a} combined in the cycloalkyl may be unsubstituted or substituted with a J group.

Specific examples include the following compounds of Formula I:

| Compound # | Compound Structure |
|---|---|
| 43 | 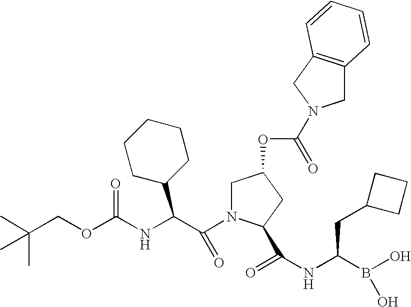 |
| 133 | 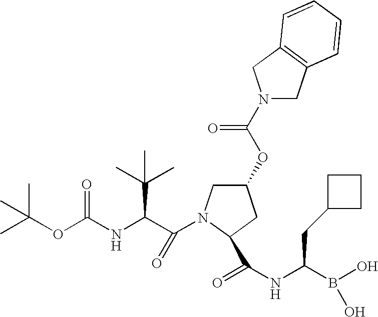 |
| 61 | 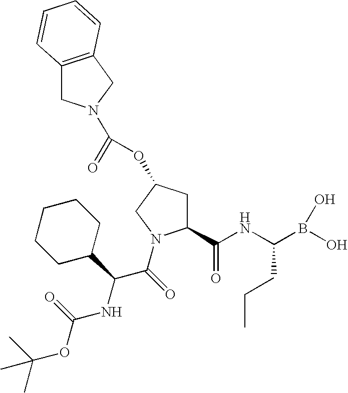 |
| 63 | 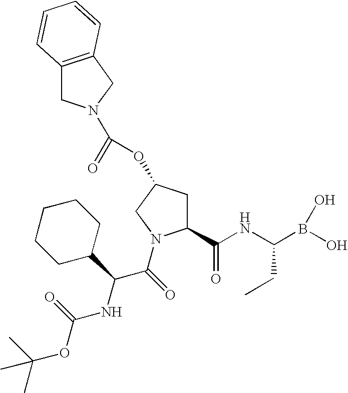 |
| 78 | 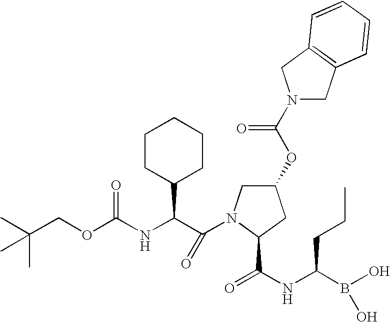 |
| 80 | 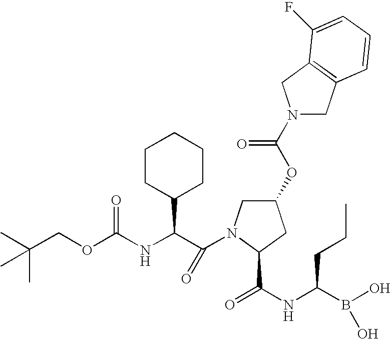 |
| 89 | 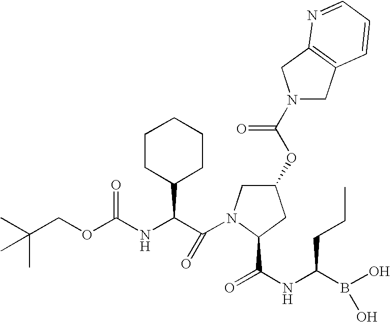 |
| 95 | 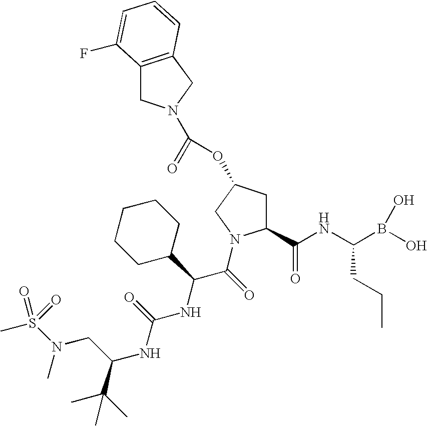 |
| 100 | 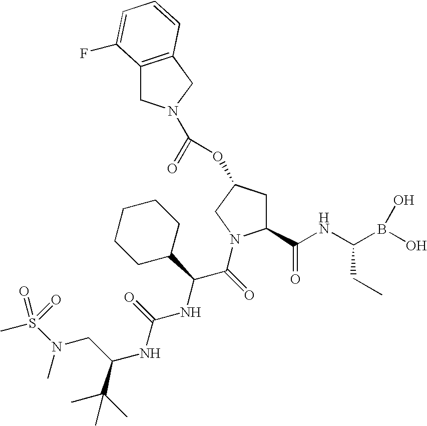 |
| 108 | 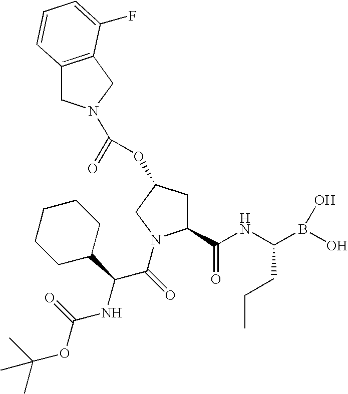 |
| 126 | 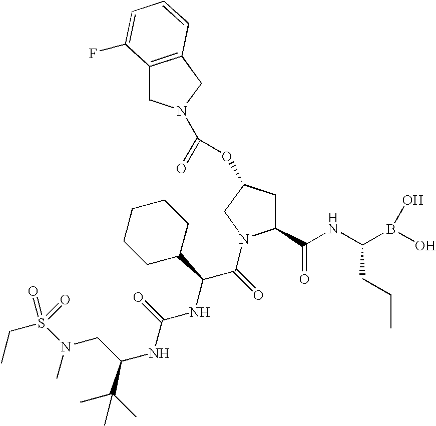 |
| 128 | 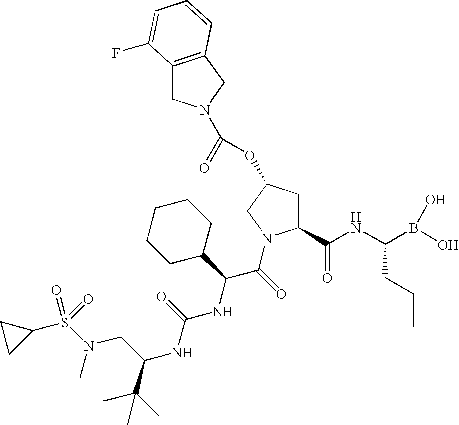 |

### Synthesis of Compounds of Formula I

Without wishing to be bound by theory, the standard nomenclature of Schechter & Berger (Biochem. Biophys. Res. Comm., 1967, 27, 157-162) regarding the identification of residues in the polypeptide substrate of serine proteases will be employed herein unless other indicia of identification are specifically provided. Within the nomenclature of Schechter & Berger, the residues of the substrate, in the direction from the N-terminal toward the C-terminal, are labeled (Pi, . . . , P3, P2, P1, P1′, P2′, Pr′ . . . , Pj), wherein cleavage is catalyzed between P1 and P1′. Within the context of this nomenclature, compounds of Formulas I can be considered as mimics of at least the tripeptide P3-Pro-P1, wherein the analog of P1 is:

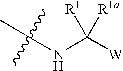

wherein W, R¹, and R^{1a} are as defined herein.

### Series A) P-1 Analogs: (S)-configuration

Compounds of the invention with a defined stereochemical configuration at the site which mimics the P1 site are available via the following scheme. As exemplified in Scheme A, a P1 analog with an absolute (S)-configuration at its α-carbon can be synthesized by the incorporation of a chiral pinanediol boronic acid protecting group that provides stereoselectivity in the following reaction and, upon hydrolysis, an enantiomerically enriched aminoboronic acid product.

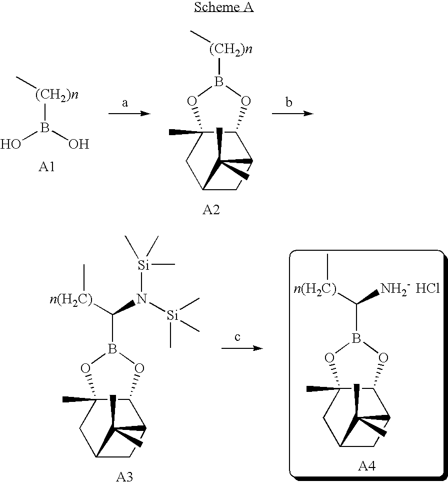

Step (a): Alkyldihydroxyborane A1 can react with (+)-pinanediol in Et₂O for 30 min to provide protected boronic acid A2. Step (b): Cmpd A2, in the presence of LiCHCl₂ at −100° C. then LiN(SiMe₃)₂ can form protected aminoalkylboronic acid A3. Step (c): In 4N HCl in dioxane at 0° C. A3 can be converted to protected aminoalkylboronic acid A4. The latter intermediate can be coupled with P2-P3 mimics as shown below to provide compounds of the invention.

### Series B) P-1 Analogs: (R)-configuration

By symmetry with respect to Scheme 1, compounds of the invention with a defined (R′)-configuration at the P1 mimic site are also available. As exemplified in Scheme B, a P1 analog with an absolute (R′)-configuration at its α-carbon can be synthesized by the incorporation of the opposite chiral pinanediol boronic acid protecting group as used in Scheme A that provides stereoselectivity in the following reaction and, upon hydrolysis, an enantiomerically enriched aminoboronic acid product of the opposite configuration to the product of Scheme A.

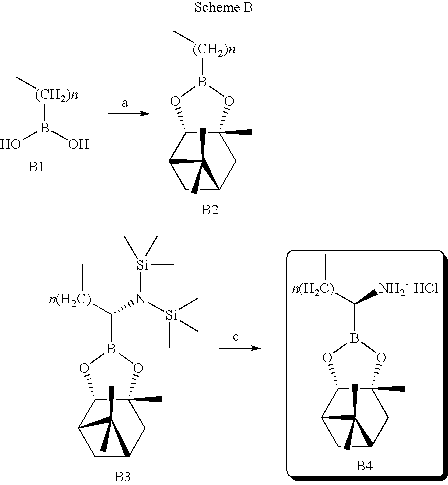

Step (a): Alkyldihydroxyborane B1 can react with (−)-pinanediol in Et₂O for 30 min to provide protected boronic acid B2. Step (b): Cmpd B2, in the presence of LiCHCl₂ at −100° C. then LiN(SiMe₃)₂ can form protected aminoalkylboronic acid B3. Step (c): In 4N HCl in dioxane at 0° C. B3 can be converted to protected aminoalkylboronic acid B4.

### Series C) Synthesis of Compounds of Formula I

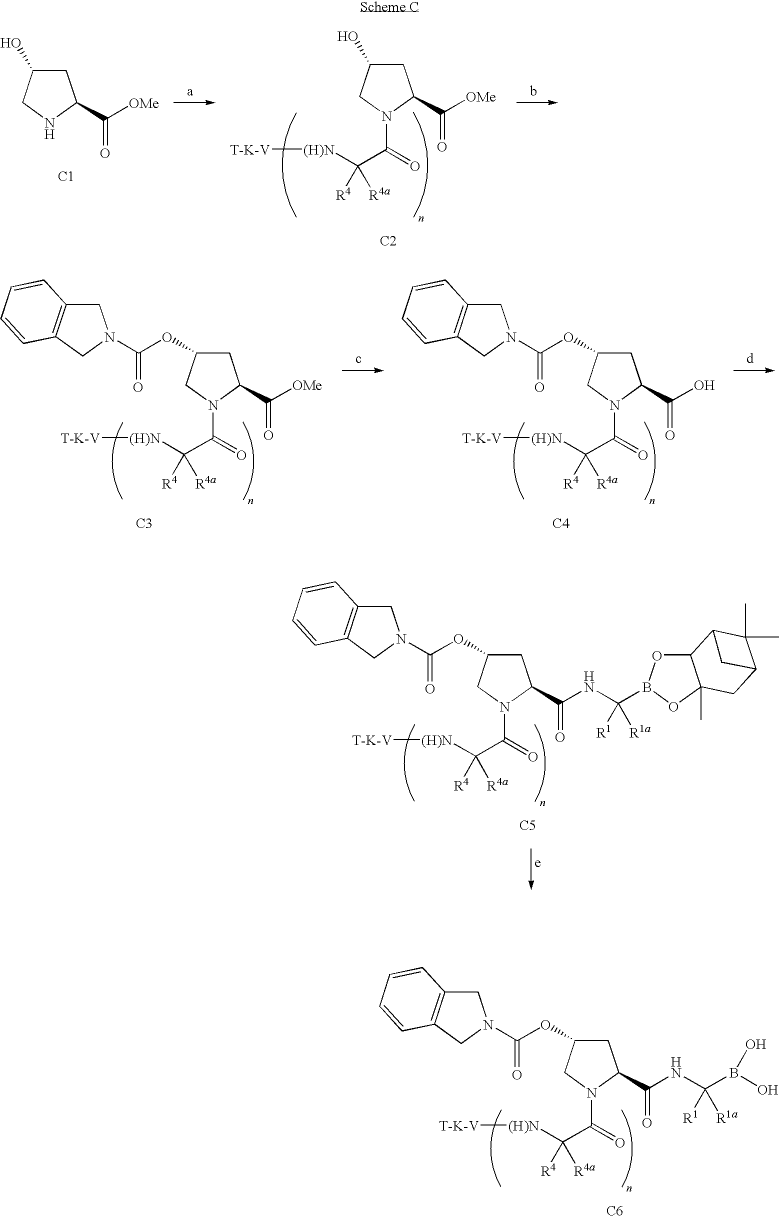

For clarity, not all possible substituents are shown in Scheme C, and a specific X-Y-Z group is indicated, but this Scheme is intended to be exemplary for all compounds of Formula I claimed herein, and other X-Y-Z groups as specified herein can be added by an analogous procedure using suitable reagents, as is well known in the art. As shown, Scheme C illustrates the preparation of a compound wherein W is —BR^{a}R^{b}, but it is understood that every other W group as specified herein can be introduced by use of that group or a suitably protected form, as is well known by the skilled artisan.

Step (a): when n=1, hydroxyproline derivative C1 is coupled with T-K-V-N(H)CR⁴R^{4a}COOH, using, e.g., EDC, HOBt, NMM in DCM/DMF to provide C2. When n=0, hydroxyproline derivative C1 is coupled in the same manner with T-K-V-OH (when V is C(O), SO, or SO₂, or V is a bond and K is C(O), SO or SO₂; when V is CH₂ or C(R¹⁰)₂, the coupling can be carried out by using reagent T-K-V-LG, wherein LG is a leaving group such as a halo, under alkaline conditions or in a dipolar aprotic solvent such as DMSO or DMF, as is well known in the art). Step (b): C2 is coupled to an N-containing heterocycle of the invention using, e.g., CDI in DCM to give the protected P3-P2 fragment, C3. However, non-heterocyclic groups could similarly be coupled, for example through an ester bond. Step (c): The C3 ester is deprotected using, e.g., LiOH in THF/water to give C4. Step (d): peptide bond formation can be achieved with the reaction of A4 or B4 with, e.g., BOP and DIEA in DCM to provide C5. However, any suitably protected analog providing W groups other than the BR^{a}R^{b} group shown can be substituted for the A4 or B4 to yield the analogous C5 bearing the alternative W group, as is well known in the art. Step (e): Deprotection of the pinanediol boronate ester C5 provides a C6 boronic acid of the invention, i.e., a compound of Formula I. Reference: Tetrahedron, 2003, 59, 579; Organometallics, 1984, 3, 1284. For example, when R¹ is cyclobutylmethyl, R⁴ is cyclohexyl, R^{4a} is hydrogen, V is —C(O)—, K is —O—, and T is t-butyl, the compound C6 of Scheme C will be seen to be Compound 15 of Example 3. Those of skill in the art will readily understand that related compounds of the invention, including compounds of Formula I bearing the alternative substituents as specified herein may be made by slight modification of this procedure. If necessary, suitable protecting groups can be used, as is well known in the art, as is described in Protective Groups in Organic Synthesis, Greene, T. W.; Wuts, P. G. M., John Wiley & Sons, New York, N.Y., (3rd Edition, 1999), which is incorporated herein by reference.

### Methods/Uses

In one aspect, the invention provides methods of inhibiting HCV NS3 protease. The methods include contacting the hepatitis C viral protease with a compound of Formula I as described herein. In other embodiments, the methods of inhibiting HCV NS3 protease include administering a compound as described herein to a subject infected with hepatitis C virus.

In another aspect, the invention provides methods for treating hepatitis C viral infection. The methods include administering to a subject in need of such treatment an effective amount of a compound of the invention as described herein. As used herein, “a compound” can refer to a single compound or a plurality of compounds. In some embodiments, the methods for treating hepatitis C viral infection include administering to a subject in need of such treatment an effective amount of a composition comprising a compound of the invention and a pharmaceutically acceptable carrier.

In another embodiment, the invention provides methods for treating hepatitis C viral infection comprising administering to a subject in need of such treatment an effective amount of a compound of the invention in combination with another medicament, such as another anti-viral agent. The term “anti-viral agent” as used herein denotes a compound which interferes with any stage of the viral life cycle to slow or prevent HCV reproduction. Representative anti-viral agents include, without limitation, NS3 protease inhibitors, INTRON-A, (interferon alfa-2b available from Schering Corporation, Kenilworth, N.J.), PEG-INTRON (peginteferon alfa-2b, available from Schering Corporation, Kenilworth, N.J.), ROFERON-A (recombinant interferon alfa-2a available Hoffmann-La Roche, Nutley, N.J.), PEGASYS (peginterferon alfa-2a available Hoffmann-La Roche, Nutley, N.J.), INFERGEN A (Schering Plough, inteferon-alpha 2B+Ribavirin), WELLFERON (interferon alpha-n1), nucleoside analogues, IRES inhibitors, NS5b inhibitors, E1 inhibitors, E2 inhibitors, IMPDH inhibitors, NS5 polymerase inhibitors and/ior NTPase/helicase inhibitors. In certain embodiments, the methods of treating HCV infection include administering to a subject in need of such treatment an effective amount of a compound of the invention in combination with another NS3 protease inhibitor. Examples of other NS3 protease inhibitors which can be administered in combination with compounds of the present invention include, without limitation, VX950 and BILN2061 (Lin C, Lin K, Luong Y, Rao B G, Wei Y Y, Brennan D L, Fulghum J R, Hsiao H M, Ma S, Maxwell J P, Cottrell K M, Perni R B, Gates C A, Kwong A D, “In Vitro Resistance Studies of Hepatitis C Virus Serine Protease Inhibitors VX950 and BILN2061”, J. Biol. Chem., 2004, 279, 17508-514).

Still other antiviral agents that may be used in conjunction with inventive compounds for the treatment of HCV infection include, but are not limited to, ribavirin (1-beta-D-ribofuranosyl-1H-1,2,-4-triazole-3-carboxamide, available from ICN Pharmaceuticals, Inc., Costa Mesa, Calif.; described in the Merck Index, entry 8365, Twelfth Edition); REBETROL.RTM. (Schering Corporation, Kenilworth, N.J.), COPEGASUS.RTM. (Hoffmann-La Roche, Nutley, N.J.); BEREFOR.RTM. (interferon alfa 2 available from Boehringer Ingelheim Pharmaceutical, Inc., Ridgefield, Conn.); SUMIFERON.RTM. (a purified blend of natural alpha interferons such as Sumiferon available from Sumitomo, Japan); ALFERON.RTM. (a mixture of natural alpha interferons made by Interferon Sciences, and available from Purdue Frederick Co., CT); .alpha.-interferon; natural alpha interferon 2a; natural alpha interferon 2b; pegylated alpha interferon 2a or 2b; consensus alpha interferon (Amgen, Inc., Newbury Park, Calif.); VIRAFERON.RTM.; INFERGEN.RTM.; REBETRON.RTM. (Schering Plough, Inteferon-alpha 2B+Ribavirin); pegylated interferon alpha (Reddy, K. R. et al. “Efficacy and Safety of Pegylated (40-kd) Interferon alpha-2a Compared with Interferon alpha-2a in Noncirrhotic Patients with Chronic Hepatitis C (Hepatology, 33, pp. 433-438 (2001); consensus interferon (Kao, J. H., et al., “Efficacy of Consensus Interferon in the Treatment of Chronic Hepatitis” J. Gastroenterol. Hepatol. 15, pp. 1418-1423 (2000); lymphoblastoid or “natural” interferon; interferon tau (Clayette, P. et al., “IFN-tau, A New Interferon Type I with Antiretroviral activity” Pathol. Biol. (Paris) 47, pp. 553-559 (1999); interleukin 2 (Davis, G. L. et al., “Future Options for the Management of Hepatitis C.” Seminars in Liver Disease, 19, pp. 103-112 (1999); Interleukin 6 (Davis et al. “Future Options for the Management of Hepatitis C.” Seminars in Liver Disease 19, pp. 103-112 (1999); interleukin 12 (Davis, G. L. et al., “Future Options for the Management of Hepatitis C.” Seminars in Liver Disease, 19, pp. 103-112 (1999); and compounds that enhance the development of type 1 helper T cell response (Davis et al., “Future Options for the Management of hepatitis C.” Seminars in Liver Disease, 19, pp. 103-112 (1999)). Also included are compounds that stimulate the synthesis of interferon in cells (Tazulakhova, E. B. et al., “Russian Experience in Screening, analysis, and Clinical Application of Novel Interferon Inducers” J. Interferon Cytokine Res., 21 pp. 65-73) including, but are not limited to, double stranded RNA, alone or in combination with tobramycin, and Imiquimod (3M Pharmaceuticals; Sauder, D. N. “Immunomodulatory and Pharmacologic Properties of Imiquimod” J. Am. Acad. Dermatol., 43 pp. S6-11 (2000).

In another embodiment, the invention provides a method for treating hepatitis C viral infection, comprising administering to a subject in need of such treatment an effective amount of a compound of the invention in combination with an anti-proliferative agent. The term “anti-proliferative agent” as used herein denotes a compound which inhibits cellular proliferation. Cellular proliferation can occur, for example without limitation, during carcinogenesis, metastasis, and immune responses. Representative anti-proliferative agents include, without limitation, 5-fluorouracil, daunomycin, mitomycin, bleomycin, dexamethasone, methotrexate, cytarabine, mercaptopunne.

In another embodiment, the invention provides a method for treating hepatitis C viral infection, comprising administering to a subject in need of such treatment an effective amount of a compound of the invention in combination with an immune modulator. The term “immune modulator” as used herein denotes a compound or composition comprising a plurality of compounds which changes any aspect of the functioning of the immune system. In this context, immune modulator includes without limitation anti-inflammatory agents and immune suppressants. Representative immune modulator include without limitation steroids, non-steroidal anti-inflammatories, COX2 inhibitors, anti-TNF compounds, anti-IL-1 compounds, interferons, methotrexate, leflunomide, cyclosporin, FK506 and combinations of any two or more thereof. Representative steroids in this context include without limitation prednisone, prednisolone, and dexamethasone. Representative non-steroidal anti-inflammatory agents in this context include without limitation ibuprofen, naproxen, diclofenac, and indomethacin. Representative COX2 inhibitors in this context include without limitation rofecoxib and celecoxib. Representative anti-TNF compounds in this context include without limitation enbrel, infliximab, and adalumimab. Representative anti-IL-1 compounds in this context include without limitation anakinra. Representative interferons include without limitation INTRON-A, (interferon alfa-2b available from Schering Corporation, Kenilworth, N.J.), PEG-INTRON (peginteferon alfa-2b, available from Schering Corporation, Kenilworth, N.J.), ROFERON-A (recombinant interferon alfa-2a available Hoffmann-La Roche, Nutley, N.J.), PEGASYS (peginterferon alfa-2a available Hoffiann-La Roche, Nutley, N.J.), WELLFERON (interferon alpha-n1). Representative immune suppressants include without limitation cyclosporin and FK506.

Compounds of the invention include mixtures of stereoisomers such as mixtures of diastereomers and/or enantiomers. In some embodiments, the compound, e.g. of Formula I, is 90 weight percent (wt %) or greater of a single diastereomer of enantiomer. In other embodiments, the compound is 92, 94, 96, 98 or even 99 wt % or more of a single diastereomer or single enantiomer.

A variety of uses of the invention compounds are possible along the lines of the various methods of treating a subject as described above. Exemplary uses of the invention methods include, without limitation, use of a compound of the invention in a medicament or for the manufacture of a medicament for treating a condition that is regulated or normalized via inhibition of the HCV NS3 serine protease.

### Biochemical methods

Fluorescence resonance energy transfer (FRET; see e.g., Heim et al., (1996) Curr. Biol. 6:178-182; Mitra et al., (1996) Gene 173:13-17; and Selvin et al., (1995) Meth. Enzymol. 246:300-345) is an exquisitely sensitive method for detecting energy transfer between two fluorophoric probes. As known in the art, such probes are given the designations “donor” and “acceptor” depending on the relative positions of the maxima in the absorption and emission spectra characterizing the probes. If the emssion spectrum of the acceptor overlaps the absorption spectrum of the donor, energy transfer can occur. Because of the known and highly non-linear relationship of energy transfer and distance between fluorophores, approximated by an inverse sixth power dependence on distance, FRET measurements correlate with distance. For example, when the probes are in proximity, such as when the probes are attached to the N- and C-termini of a peptide substrate, and the sample is illuminated in a spectrofluorometer, resonance energy can be transferred from one excited probe to the other resulting in observable signal. Upon scission of the peptide linking the probes, the average distance between probes increases such that energy transfer between donor and accept probe is not observed. As a result, the degree of hydrolysis of the peptide substrate, and the level of activity of the protease catalyzing hydrolysis of the peptide substrate, can be quantitated. Accordingly, using methods known in the arts of chemical and biochemical kinetics and equilibria, the effect of inhibitor on protease activity can be quantitated.

### Compositions and Combination Treatments

### A. Compositions

Another aspect of the invention provides compositions of the compounds of the invention, alone or in combination with another NS3 protease inhibitor or another type of antiviral agent and/or another type of therapeutic agent. As set forth herein, compounds of the invention include stereoisomers, tautomers, solvates, prodrugs, salts, pharmaceutically acceptable salts and mixtures thereof. Compositions containing a compound of the invention may be prepared by conventional techniques, e.g. as described in Remington: The Science and Practice of Pharmacy, 19th Ed., 1995. The compositions may appear in conventional forms, for example capsules, tablets, aerosols, solutions, suspensions or topical applications.

Typical compositions include a compound of the invention which inhibits the enzymatic activity of the HCV NS3 protease, and a pharmaceutically acceptable excipient which may be a carrier or a diluent. For example, the active compound will usually be mixed with a carrier, or diluted by a carrier, or enclosed within a carrier which may be in the form of an ampoule, capsule, sachet, paper, or other container. When the active compound is mixed with a carrier, or when the carrier serves as a diluent, it may be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent may include any sustained release material known in the art, such as glyceryl monostearate or glyceryl distearate, alone or mixed with a wax.

The formulations can be mixed with auxiliary agents which do not deleteriously react with the active compounds. Such additives can include wetting agents, emulsifying and suspending agents, salt for influencing osmotic pressure, buffers and/or coloring substances preserving agents, sweetening agents or flavoring agents. The compositions can also be sterilized if desired.

The route of administration may be any route which effectively transports the active compound of the invention which inhibits the enzymatic activity of the HCV NS3 protease to the appropriate or desired site of action, such as oral, nasal, pulmonary, buccal, subdermal, intradermal, transdermal or parenteral, e.g., rectal, depot, subcutaneous, intravenous, intraurethral, intramuscular, intranasal, ophthalmic solution or an ointment, the oral route being preferred.

If a solid carrier is used for oral administration, the preparation may be tabletted, placed in a hard gelatin capsule in powder or pellet form or it can be in the form of a troche or lozenge. If a liquid carrier is used, the preparation may be in the form of a syrup, emulsion, soft gelatin capsule or sterile injectable liquid such as an aqueous or non-aqueous liquid suspension or solution.

Injectable dosage forms generally include aqueous suspensions or oil suspensions which may be prepared using a suitable dispersant or wetting agent and a suspending agent Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Preferably, the oil or fatty acid is non-volatile, including natural or synthetic oils, fatty acids, mono-, di- or tri-glycerides.

For injection, the formulation may also be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these. The compounds may be formulated for parenteral administration by injection such as by bolus injection or continuous infusion. A unit dosage form for injection may be in ampoules or in multi-dose containers.

The formulations of the invention may be designed to provide quick, sustained, or delayed release of the active ingredient after administration to the patient by employing procedures well known in the art. Thus, the formulations may also be formulated for controlled release or for slow release.

Compositions contemplated by the present invention may comprise, for example, micelles or liposomes, or some other encapsulated form, or may be administered in an extended release form to provide a prolonged storage and/or delivery effect. Therefore, the formulations may be compressed into pellets or cylinders and implanted intramuscularly or subcutaneously as depot injections or as implants such as stents. Such implants may employ known inert materials such as silicones and biodegradable polymers, e.g., polylactide-polyglycolide. Examples of other biodegradable polymers include poly(orthoesters) and poly(anhydrides).

For nasal administration, the preparation may contain a compound of the invention which inhibits the enzymatic activity of the HCV NS3 protease, dissolved or suspended in a liquid carrier, preferably an aqueous carrier, for aerosol application. The carrier may contain additives such as solubilizing agents, e.g., propylene glycol, surfactants, absorption enhancers such as lecithin (phosphatidylcholine) or cyclodextrin, or preservatives such as parabenes.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet that may be prepared by conventional tabletting techniques may contain:

Core:

| | |
|---|---|
| Active compound (as free compound or salt thereof) | 250 mg |
| Colloidal silicon dioxide (Aerosil) ® | 1.5 mg |
| Cellulose, microcryst. (Avicel) ® | 70 mg |
| Modified cellulose gum (Ac-Di-Sol) ® | 7.5 mg |
| Magnesium stearate | Ad. |

Coating:

| | |
|---|---|
| HPMC approx. | 9 mg |
| *Mywacett 9-40 T approx. | 0.9 mg |

*Acylated monoglyceride used as plasticizer for film coating.

A typical capsule for oral administration contains compounds of the invention (250 mg), lactose (75 mg) and magnesium stearate (15 mg). The mixture is passed through a 60 mesh sieve and packed into a No. 1 gelatin capsule. A typical injectable preparation is produced by aseptically placing 250 mg of compounds of the invention into a vial, aseptically freeze-drying and sealing. For use, the contents of the vial are mixed with 2 mL of sterile physiological saline, to produce an injectable preparation.

The compounds of the invention may be administered to a mammal, especially a human in need of such treatment, prevention, elimination, alleviation or amelioration of the various diseases as mentioned above, e.g., HCV infection. Such mammals include also animals, both domestic animals, e.g. household pets, farm animals, and non-domestic animals such as wildlife.

The compounds of the invention are effective over a wide dosage range. For example, in the treatment of adult humans, dosages from about 0.5 to about 5000 mg, preferably from about 1 to about 2000 mg, more preferably from about 2 to about 2000 mg, and most preferably from about 10 to about 1000 mg per day may be used. In choosing a regimen for patients it may frequently be necessary to begin with a higher dosage and when the condition is under control to reduce the dosage. The exact dosage will depend upon the activity of the compound, mode of administration, on the therapy desired, form in which administered, the subject to be treated and the body weight of the subject to be treated, and the preference and experience of the physician or veterinarian in charge. HCV NS3 protease inhibitor activity of the compounds of the invention may be determined by use of an in vitro assay system which measures the potentiation of inhibition of the HCV NS3 protease. Inhibition constants (i.e., K₁ or IC₅₀ values as known in the art) for the HCV NS3 protease inhibitors of the invention may be determined by the method described in the Examples.

Generally, the compounds of the invention are dispensed in unit dosage form comprising from about 0.5 mg to about 5000 mg of active ingredient together with a pharmaceutically acceptable carrier per unit dosage.

Usually, dosage forms suitable for oral, nasal, pulmonal or transdermal administration comprise from about 500 μg to about 5000 mg, preferably from about 1 to about 2000 mg, more preferably from about 2 to about 2000 mg, and most preferably from about 10 to about 1000 mg, of the compounds admixed with a pharmaceutically acceptable carrier or diluent.

The invention also encompasses prodrugs of a compound of the invention which on administration undergo chemical conversion by metabolic or other physiological processes before becoming active pharmacological substances. Conversion by metabolic or other physiological processes includes without limitation enzymatic (e.g, specific enzymatically catalyzed) and non-enzymatic (e.g., general or specific acid or base induced) chemical transformation of the prodrug into the active pharmacological substance. In general, such prodrugs will be functional derivatives of a compound of the invention which are readily convertible in vivo into a compound of the invention. Conventional procedures for the selection and preparation of suitable prodrug derivatives are described, for example, in Design of Prodrugs, ed. H. Bundgaard, Elsevier, 1985.

In another aspect, there are provided methods of making a composition of a compound described herein comprising formulating a compound of the invention with a pharmaceutically acceptable carrier or diluent. In some embodiments, the pharmaceutically acceptable carrier or diluent is suitable for oral administration. In some such embodiments, the methods may further comprise the step of formulating the composition into a tablet or capsule. In other embodiments, the pharmaceutically acceptable carrier or diluent is suitable for parenteral administration. In some such embodiments, the methods further comprise the step of lyophilizing the composition to form a lyophilized preparation.

### B. Combinations

The compounds of the invention may be used in combination with i) one or more other NS3 protease inhibitors and/or ii) one or more other types of antiviral agents (employed to treat viral infection and related diseases) and/or one or more other types of therapeutic agents which may be administered orally in the same dosage form, in a separate oral dosage form (e.g., sequentially or non-sequentially) or by injection together or separately (e.g., sequentially or non-sequentially).

Accordingly, in another aspect the invention provides combinations, comprising:
a) a compound of the invention as described herein; and
b) one or more compounds comprising:
   i) other compounds of the present invention
   ii) anti-viral agents including, but not limited to, other NS3 protease inhibitors
   iii) anti-proliferative agents
   iv) immune modulators.

Combinations of the invention include mixtures of compounds from (a) and (b) in a single formulation and compounds from (a) and (b) as separate formulations. Some combinations of the invention may be packaged as separate formulations in a kit. In some embodiments, two or more compounds from (b) are formulated together while a compound of the invention is formulated separately.

Combinations of the invention can further comprise a pharmaceutically acceptable carrier. In some embodiments, the compound of the invention is 90 wt % or more of a single diastereomer or single enantiomer. Alternatively, the compound of the invention can be 91, 92, 93, 94, 95, 96, 97, 98, or 99 wt % or more of a single diastereomer or single enantiomer.

The dosages and formulations for the other antiviral agent to be employed, where applicable, will be as set out in the latest edition of the Physicians' Desk Reference.

In carrying out the methods of the invention, a composition may be employed containing the compounds of the invention, with or without another antiviral agent and/or other type therapeutic agent, in association with a pharmaceutical vehicle or diluent. The composition can be formulated employing conventional solid or liquid vehicles or diluents and pharmaceutical additives of a type appropriate to the mode of desired administration. The compounds can be administered to mammalian species including humans, monkeys, dogs, etc. by an oral route, for example, in the form of tablets, capsules, granules or powders, or they can be administered by a parenteral route in the form of injectable preparations. The dose for adult humans is preferably between 10 and 1,000 mg per day, which can be administered in a single dose or in the form of individual doses from 1-4 times per day.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present invention, thus generally described, will be understood more readily by reference to the following examples, which are provided by way of illustration and are not intended to be limiting of the present invention.

### EXAMPLES

### Exemplary Structures of the Invention

| Compound | Structure | LCMS |
|---|---|---|
| 44 | 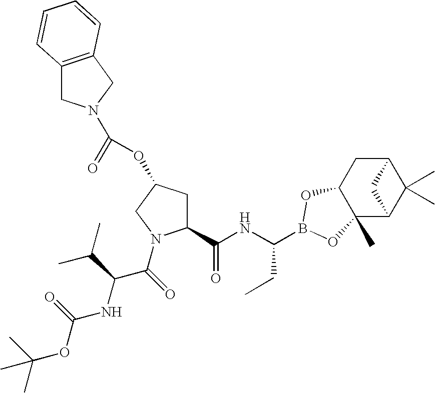 | 695(M + 1) |
| 45 | 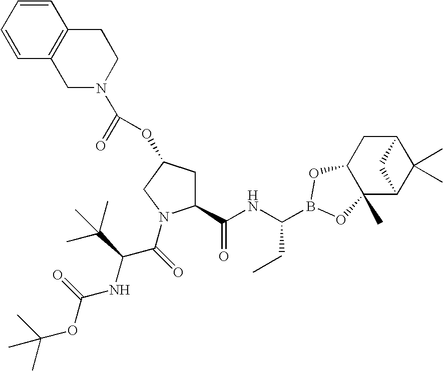 | 723(M + 1) |
| 46 | 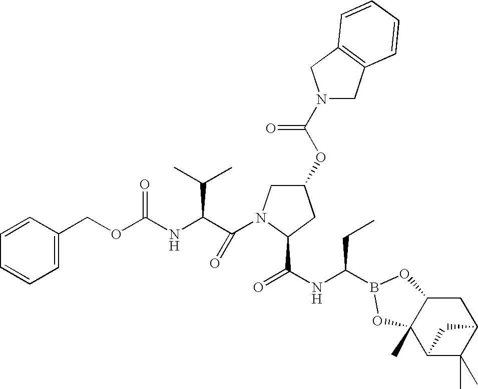 | 729(M + 1) |
| 47 | 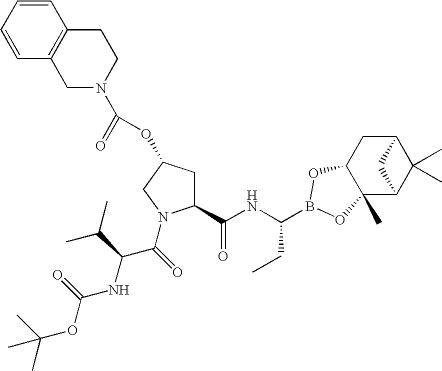 | 709(M + 1) |
| 48 | 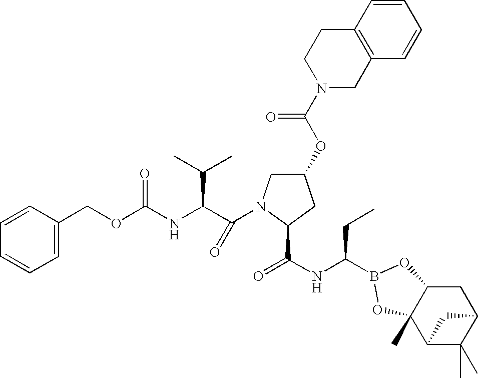 | 744(M + 1) |
| 49 | 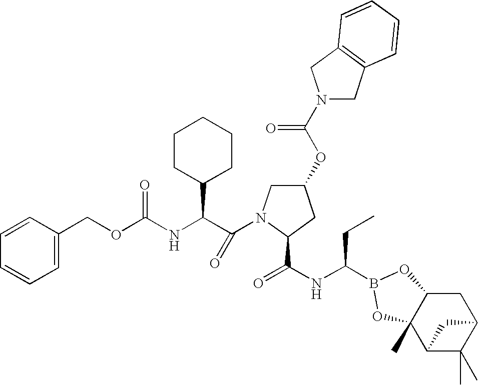 | 770(M + 1) |
| 50 | 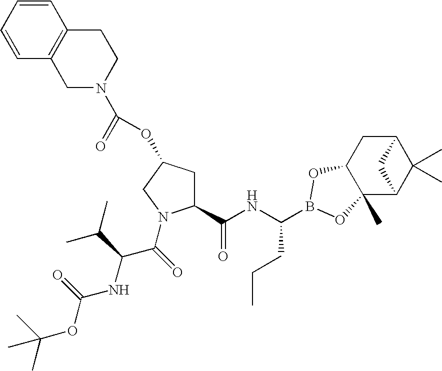 | 723(M + 1) |
| 51 | 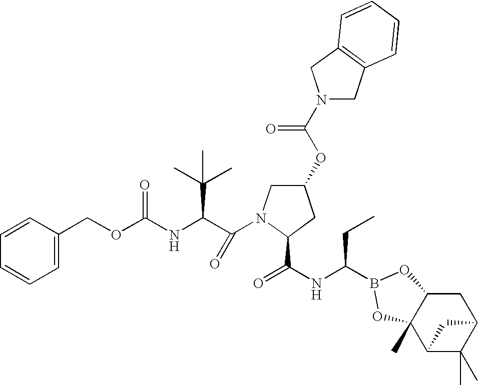 | 744(M + 1) |
| 52 | 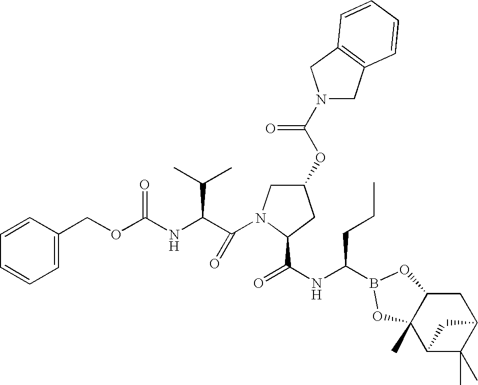 | 743(M + 1) |
| 53 | 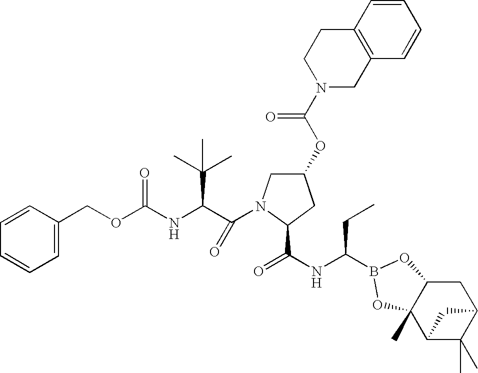 | 758(M + 1) |
| 54 | 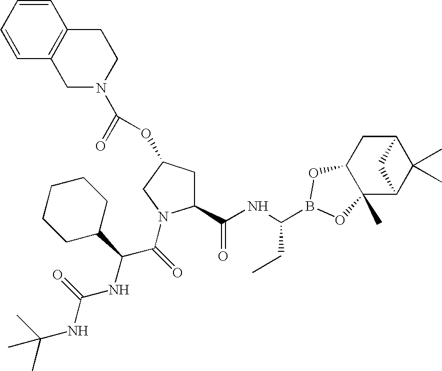 | 748(M + 1) |
| 55 | 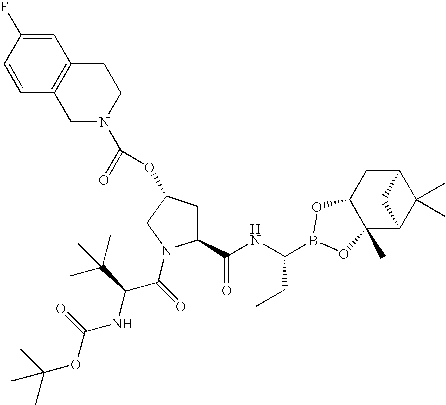 | 741(M + 1) |
| 56 | 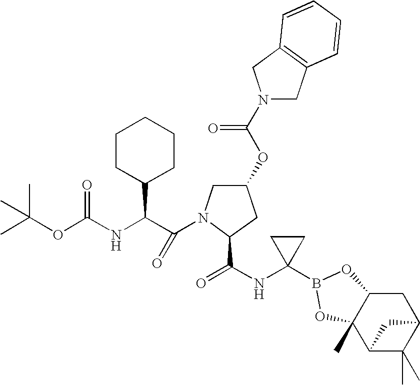 | 733(M + 1) |
| 57 | 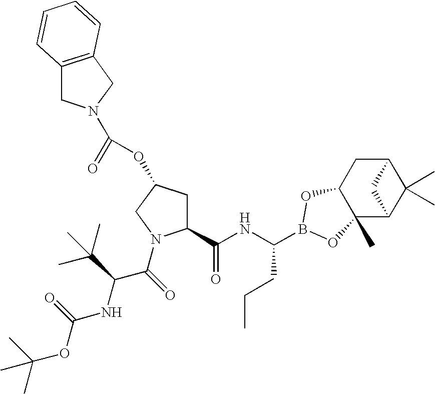 | 723(M + 1) |
| 58 | 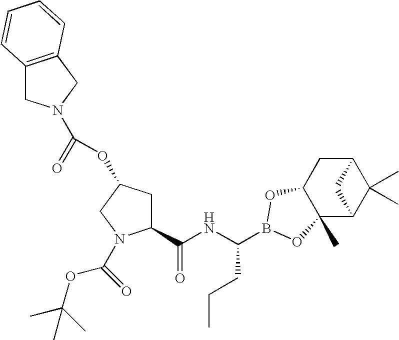 | 610(M + 1) |
| 59 | 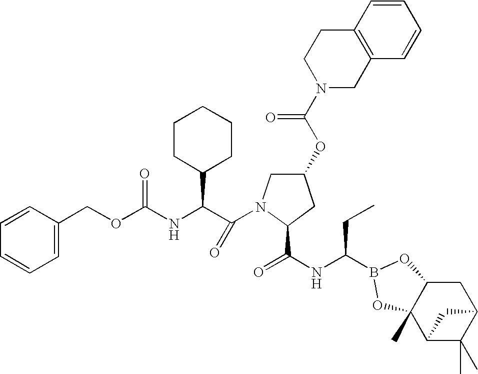 | 783(M + 1) |
| 60 | 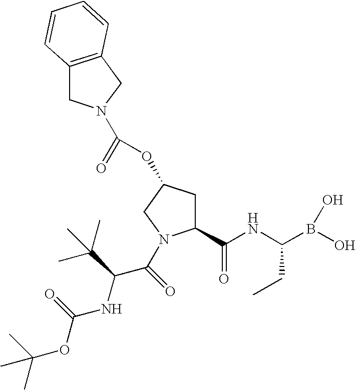 | 597(M + 23) |
| 61 | 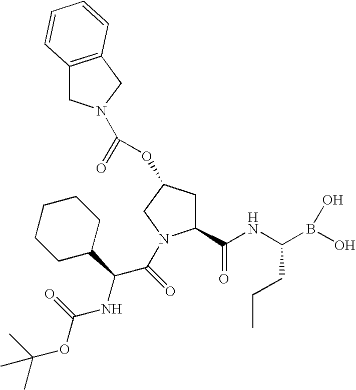 | 597(M − 17) |
| 62 | 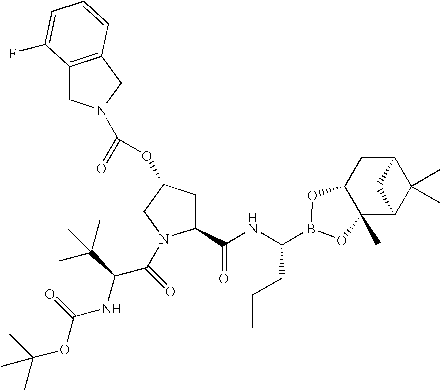 | 741(M + 1) |
| 63 | 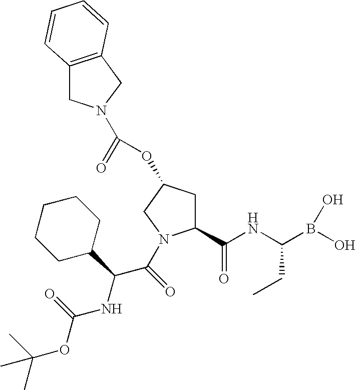 | 623(M + 23) |
| 64 | 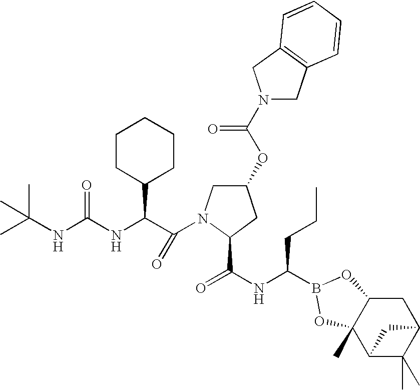 | 748(M + 1) |
| 65 | 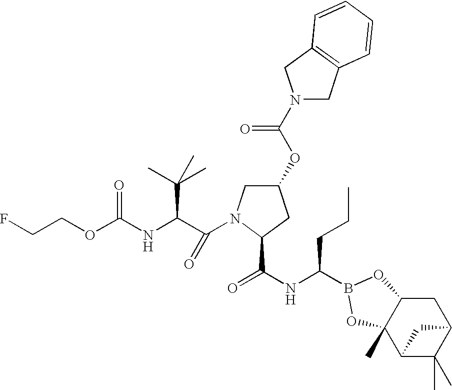 | 713(M + 1) |
| 66 | 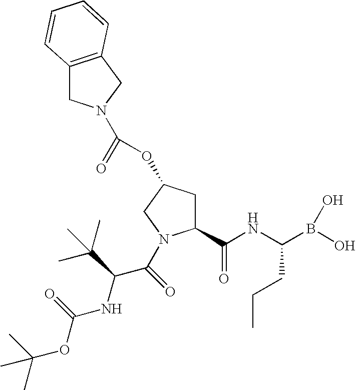 | 571(M − 17) |
| 67 | 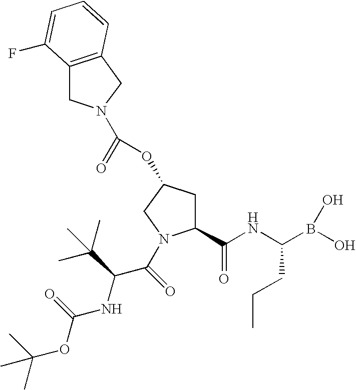 | 589(M − 17) |
| 68 | 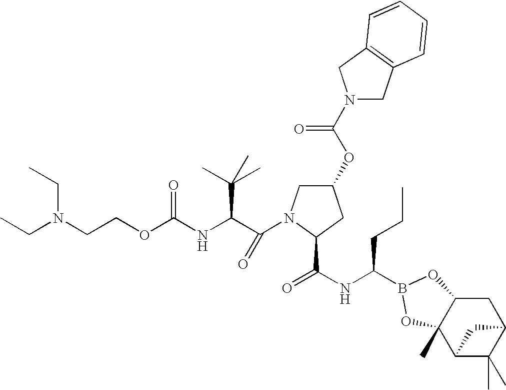 | 766(M + 1) |
| 69 | 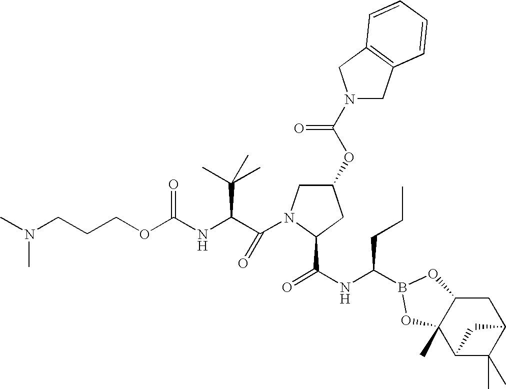 | 752(M + 1) |
| 70 | 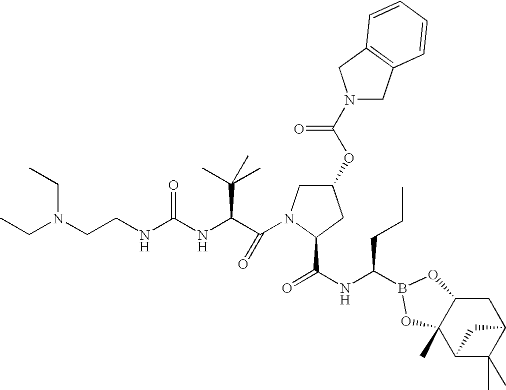 | 765(M + 1) |
| 71 | 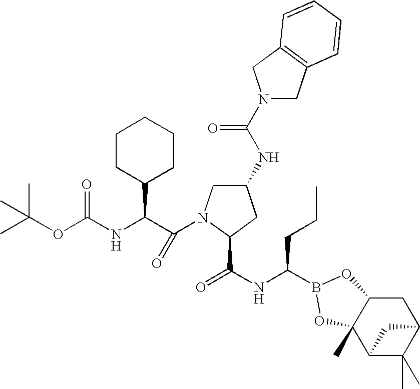 | 748(M + 1) |
| 72 | 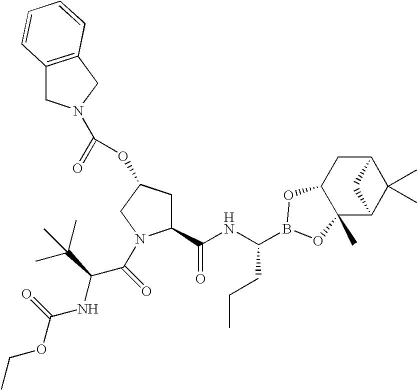 | 695(M + 1) |
| 73 | 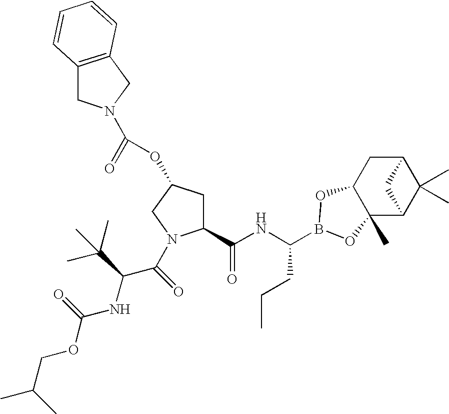 | 723(M + 1) |
| 74 | 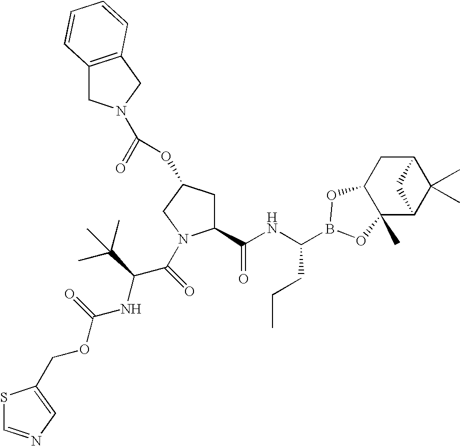 | 764(M + 1) |
| 75 | 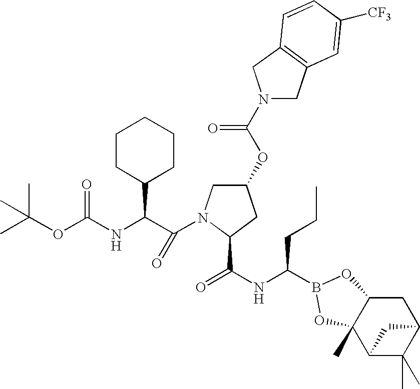 | 817(M + 1) |
| 76 | 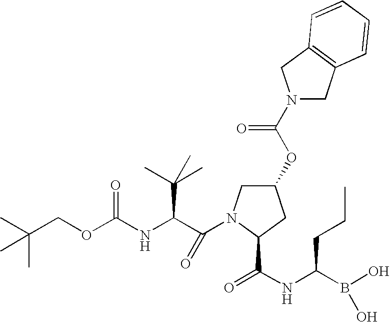 | 587(M − 17) |
| 77 | 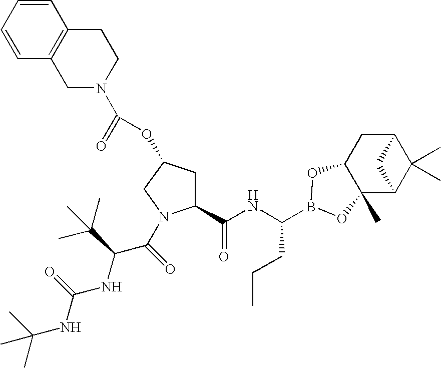 | 736(M + 1) |
| 78 | 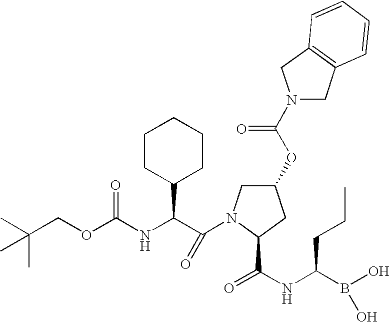 | 611(M − 17) |
| 79 | 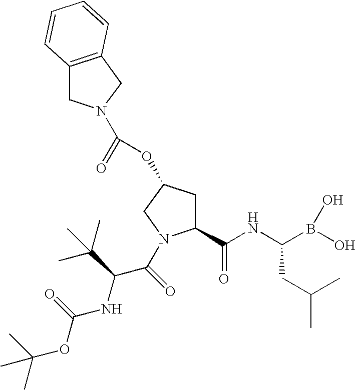 | 625(M + 23) |
| 80 | 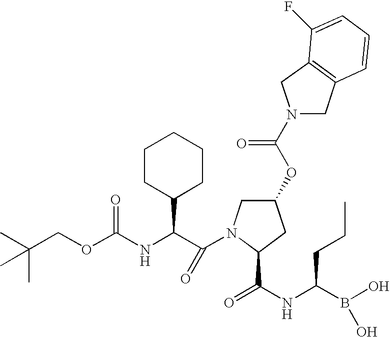 | 629(M − 17) |
| 81 | 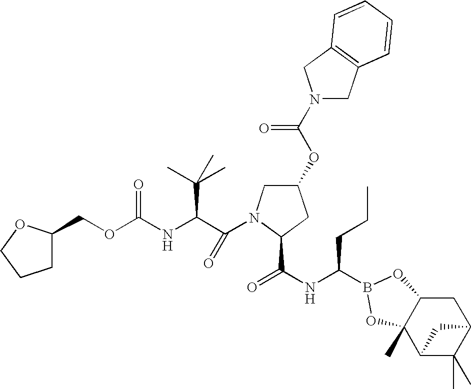 | 751(M + 1) |
| 82 | 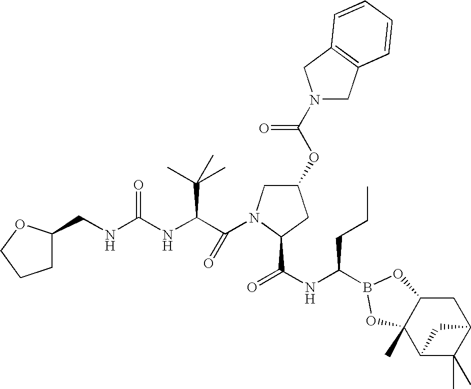 | 750(M + 1) |
| 83 | 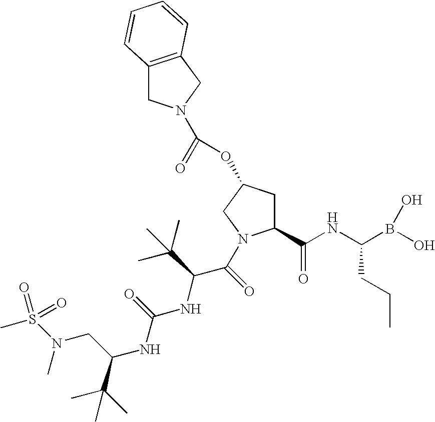 | 705(M − 17) |
| 84 | 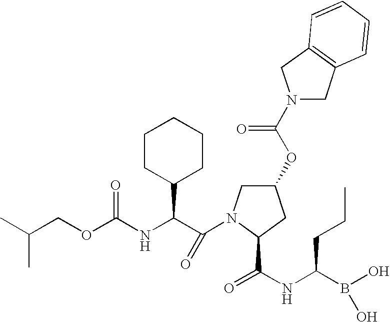 | 597(M − 17) |
| 85 | 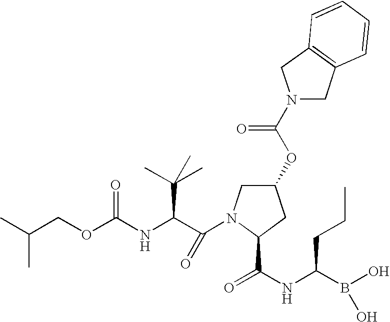 | 571(M − 17) |
| 86 | 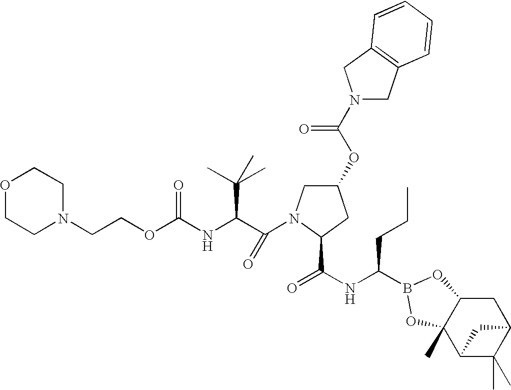 | 780(M + 1) |
| 87 | 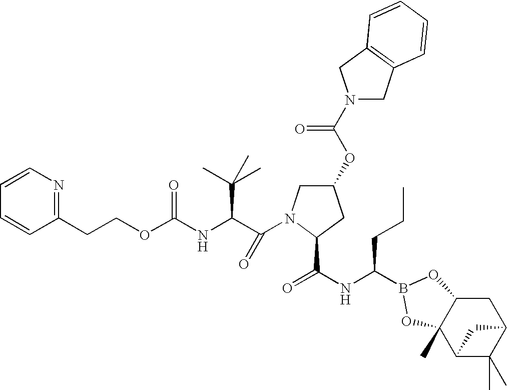 | 772(M + 1) |
| 88 | 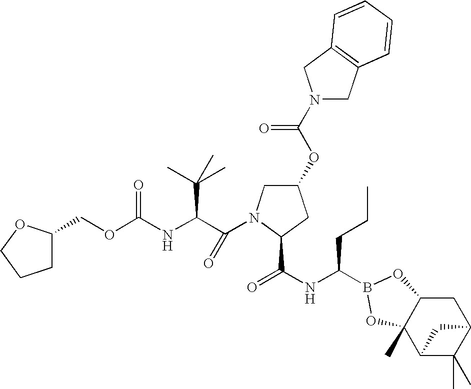 | 751(M + 1) |
| 89 | 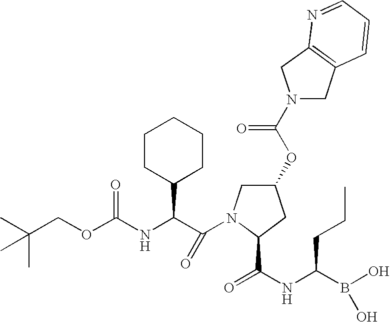 | 612(M − 17) |
| 90 | 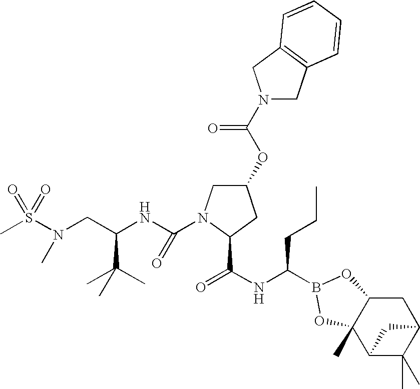 | 744(M + 1) |
| 91 | 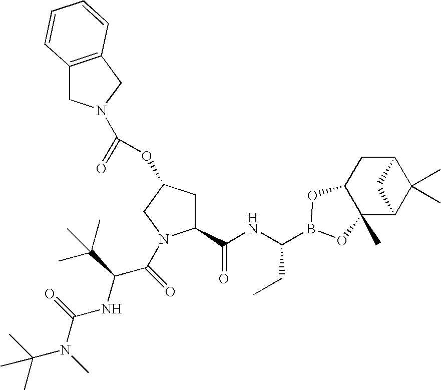 | 722(M + 1) |
| 92 | 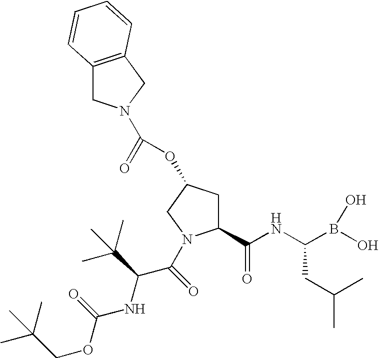 | 599(M − 17) |
| 93 | 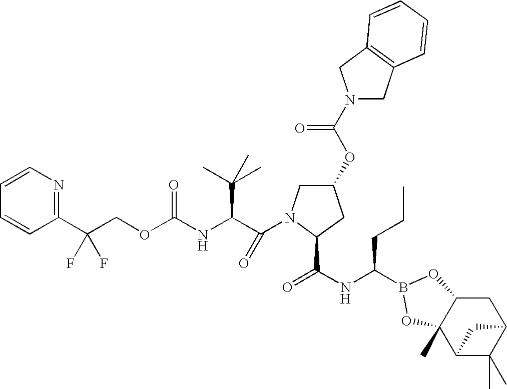 | 808(M + 1) |
| 94 | 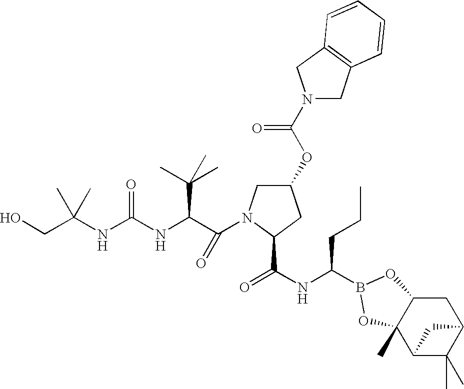 | 738(M + 1) |
| 95 | 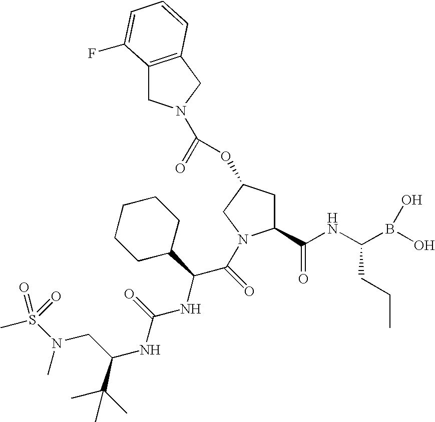 | 749(M − 17) |
| 96 | 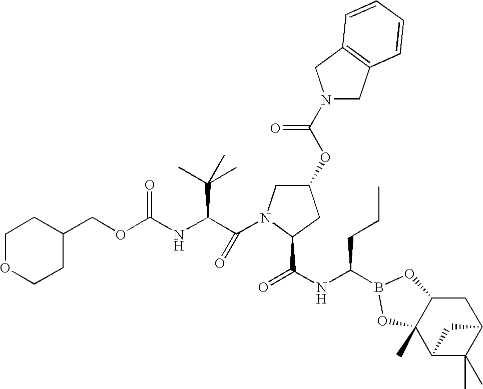 | 765(M + 1) |
| 97 | 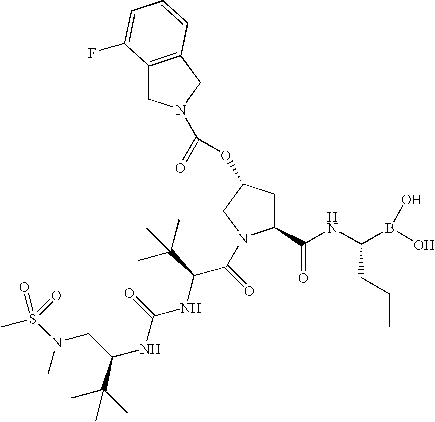 | 723(M − 17) |
| 98 | 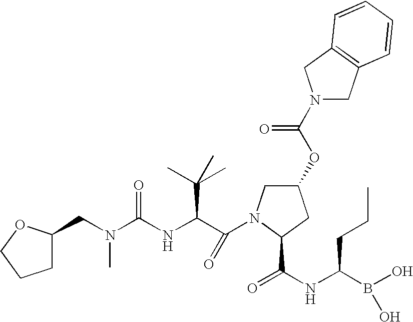 | 630(M + 1) |
| 99 | 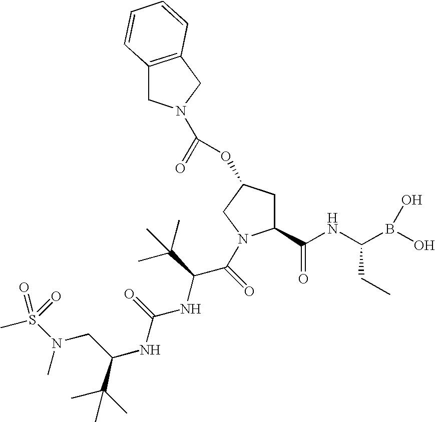 | 691(M − 17) |
| 100 | 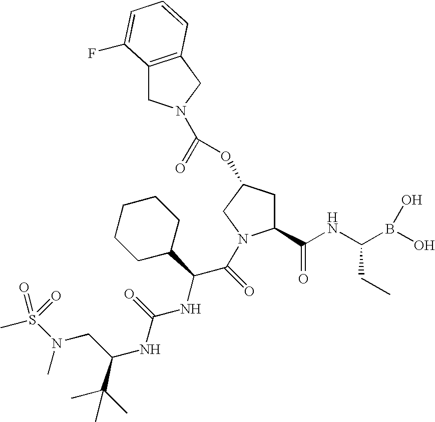 | 735(M − 17) |
| 101 | 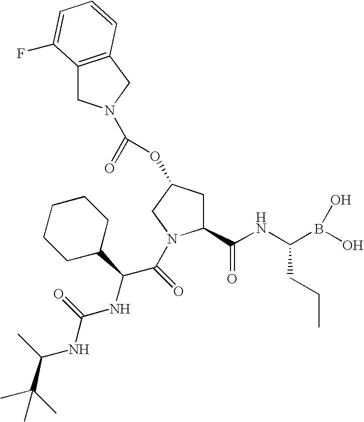 | 642(M − 17) |
| 102 | 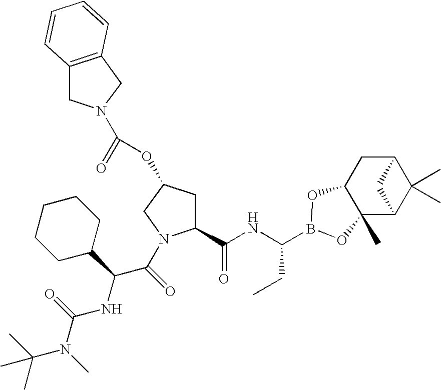 | 748(M + 1) |
| 103 | 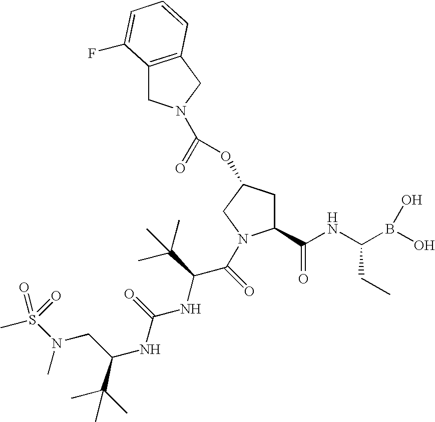 | 709(M − 17) |
| 104 | 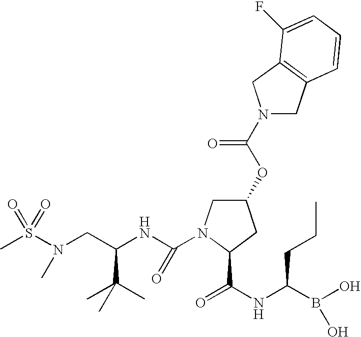 | 610(M − 17) |
| 105 | 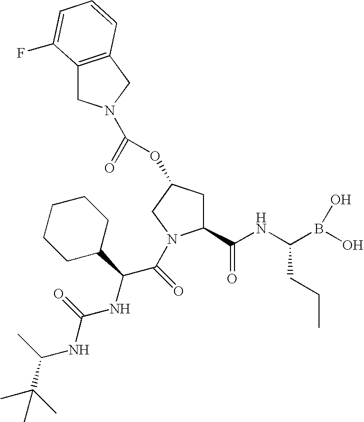 | 642(M − 17) |
| 106 | 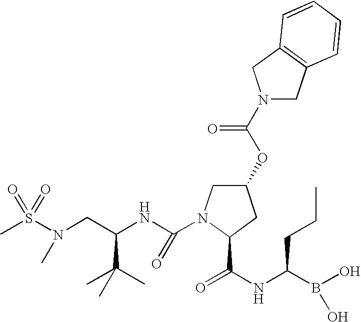 | 592(M − 17) |
| 107 | 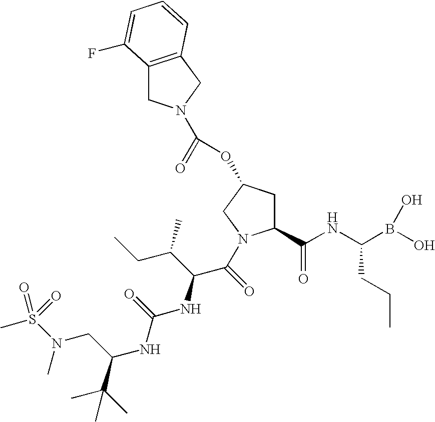 | 723(M − 17) |
| 108 | 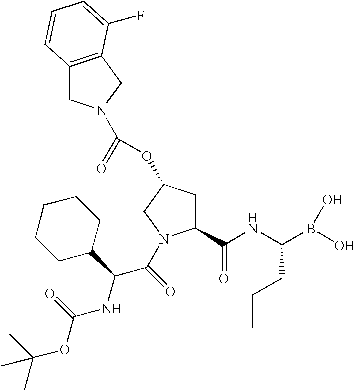 | 615(M − 17) |
| 109 | 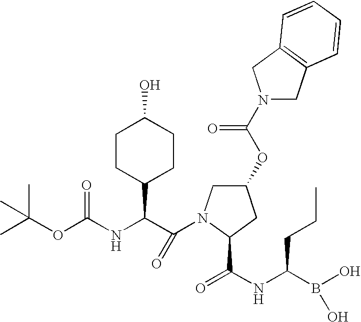 | 613(M − 17) |
| 110 | 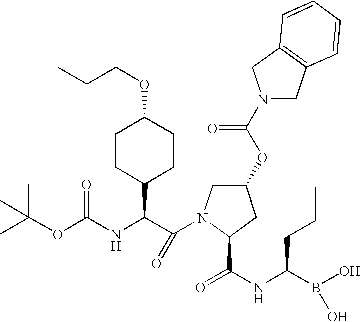 | 655(M − 17) |
| 111 | 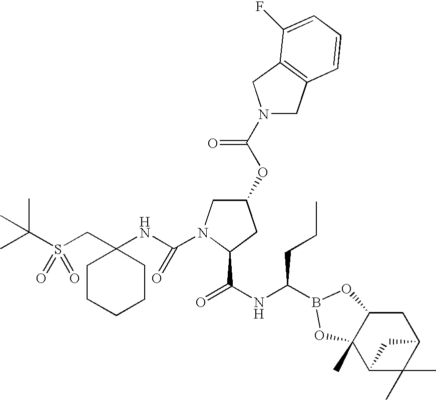 | 787(M + 1) |
| 112 | 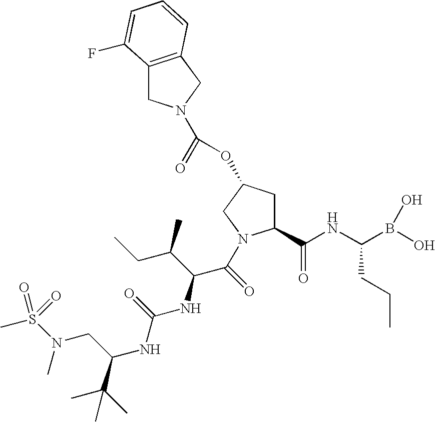 | 723(M − 17) |
| 113 | 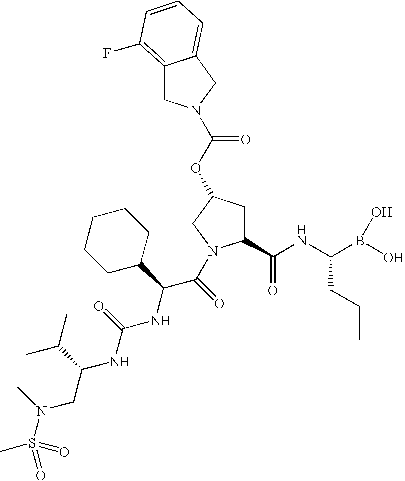 | 735(M − 17) |
| 114 | 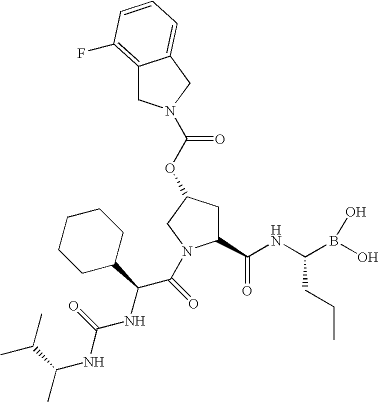 | 628(M − 17) |
| 115 | 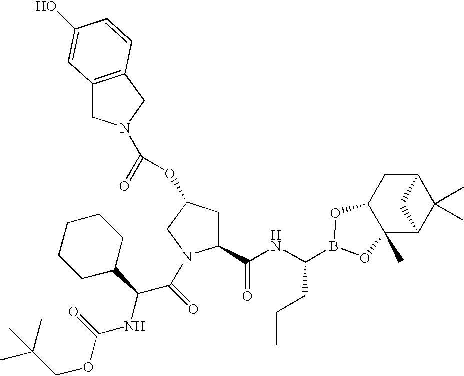 | 779(M + 1) |
| 116 | 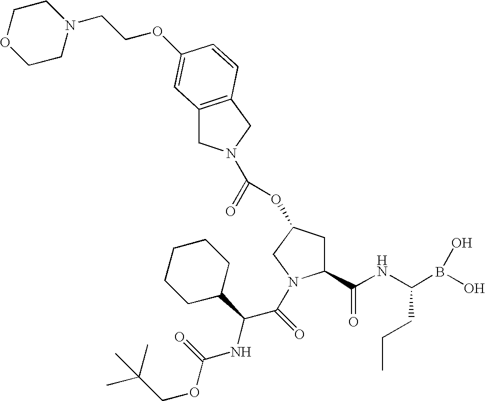 | 740(M − 17) |
| 117 | 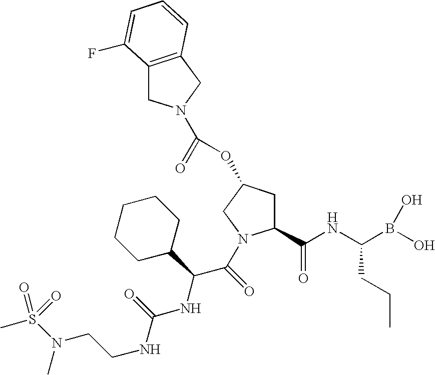 | 693(M − 17) |
| 118 | 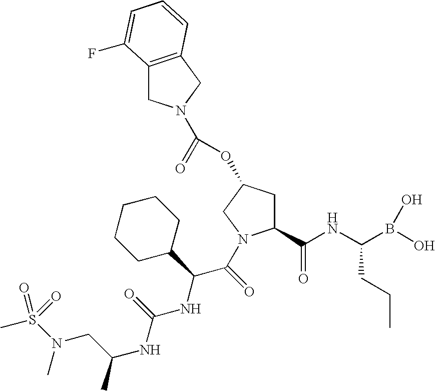 | 707(M − 17) |
| 119 | 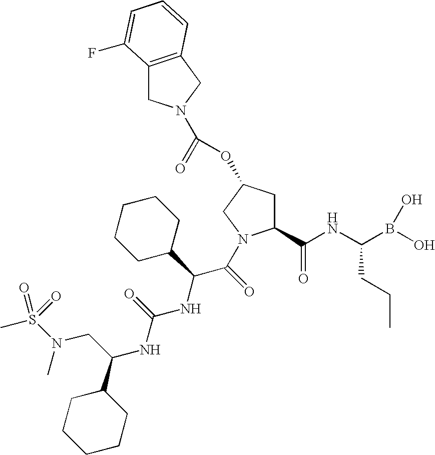 | 775(M − 17) |
| 120 | 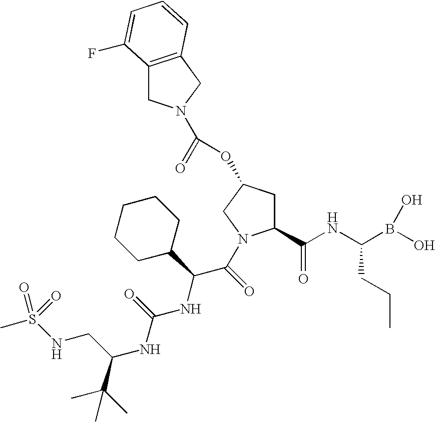 | 735(M − 17) |
| 121 | 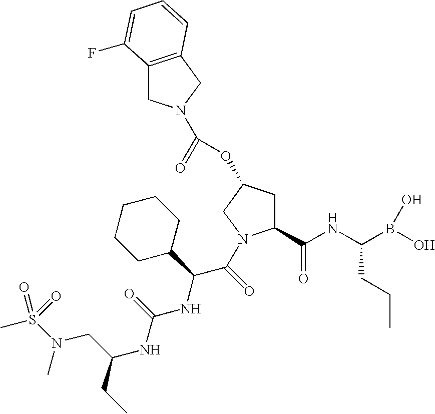 | 721(M − 17) |
| 122 | 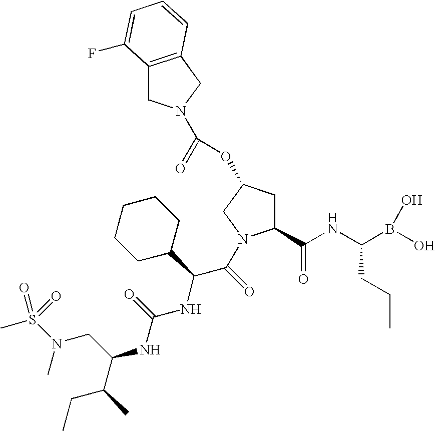 | 749(M − 17) |
| 123 | 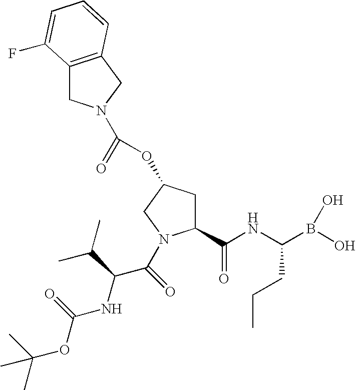 | 575(M − 17) |
| 124 | 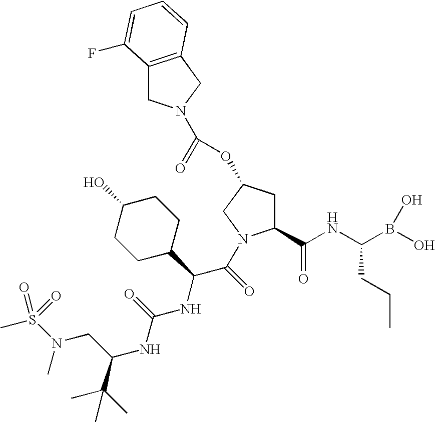 | 783(M + 1) |
| 125 | 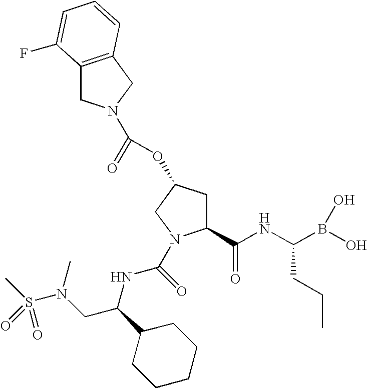 | 636(M − 17) |
| 126 | 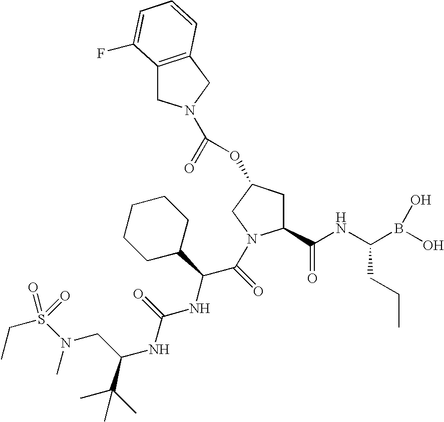 | 763(M − 17) |
| 127 | 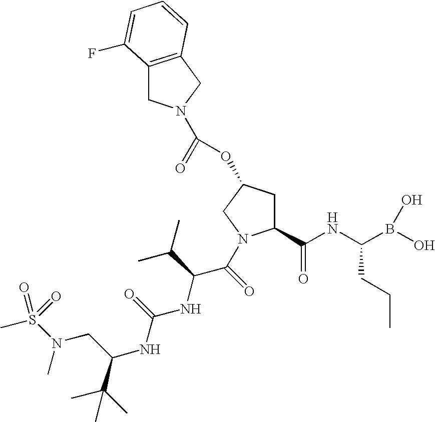 | 709(M − 17) |
| 128 | 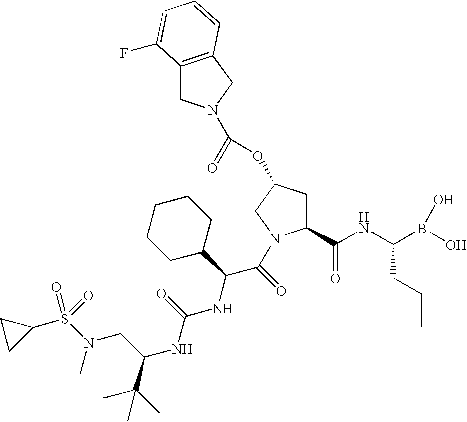 | 775(M − 17) |
| 129 | 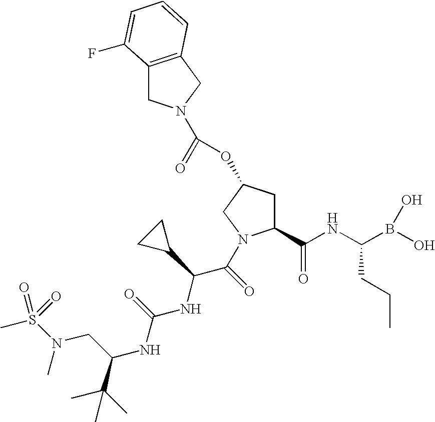 | 707(M − 17) |
| 130 | 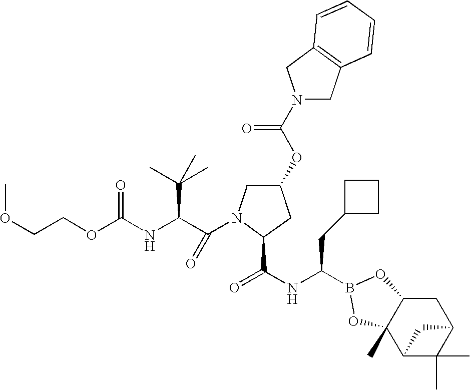 | 752(M + 1) |
| 131 | 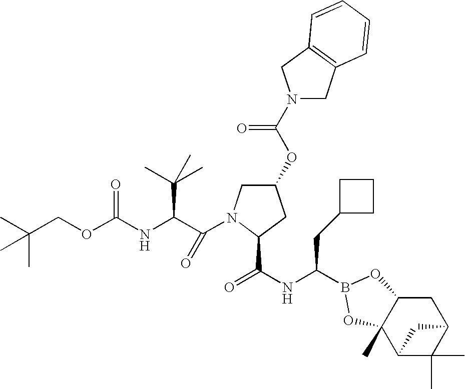 | 763(M + 1) |
| 132 | 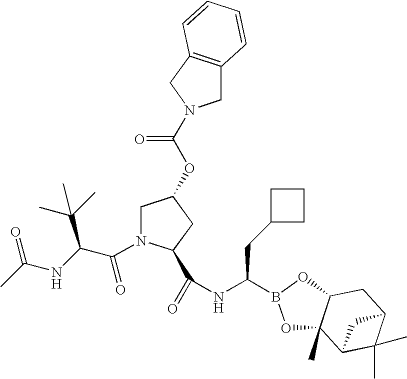 | 691(M + 1) |
| 43 | 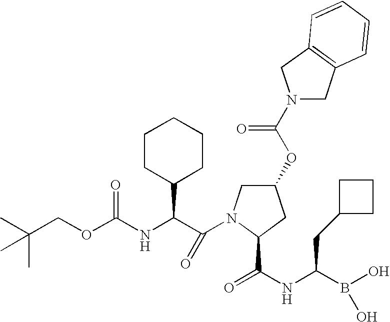 | 637(M − 17) |
| 133 | 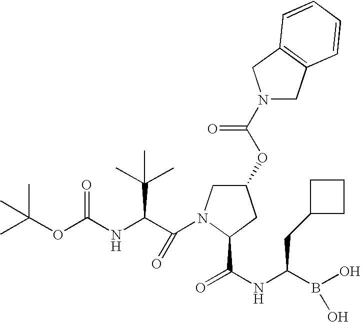 | 598(M − 17) |

The following abbreviations are used throughout this document.
BOP Benzotriazol-1-yl-oxy-tris(dimethylamino) phosphonium hexafluorophosphate
CDI Carbonyl diimidazole
DCM Dichloromethane
DIEA, ⁱPr₂EtN N,N-Diisoproylethylamine
DMF N,N-Dimethylformamide
DMSO Dimethylsulfoxide
EDC 1-Ethyl-3-[3-dimethylaminopropyl]carbodiimide hydrochloride
HOBt Hydroxybenzotriazole
MS Mass spectroscopy
MeOH Methanol
NMM N-Methylmorpholine
THF Tetrahydrofuran

### Preparative Example 1

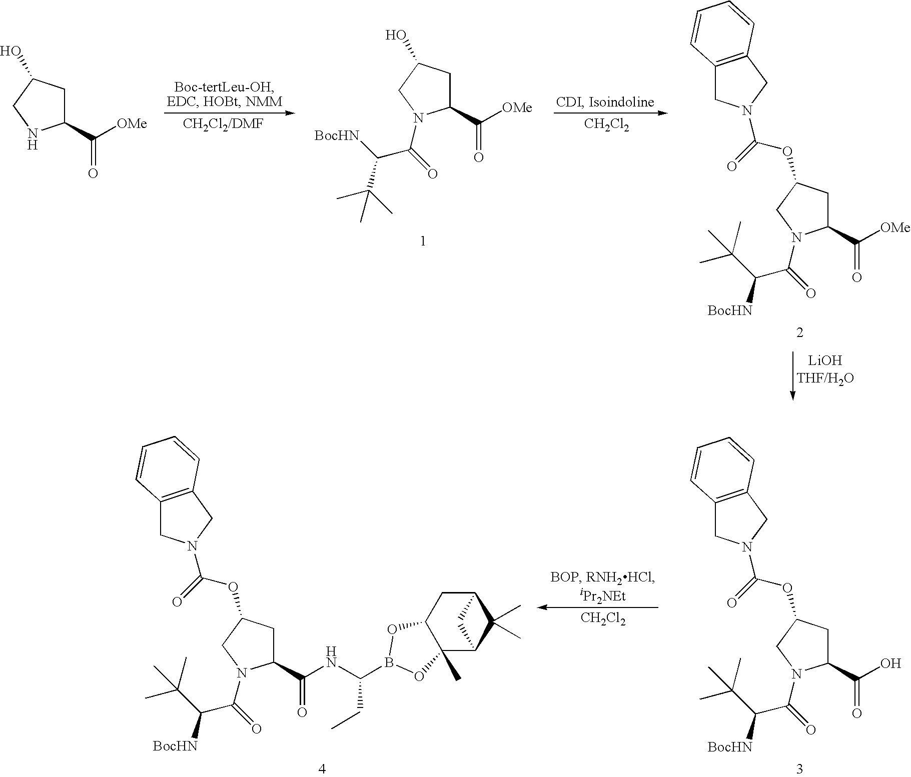

### 1-(2-tert-Butoxycarbonylamino-3,3-dimethyl-butyryl)-4-hydroxy-pyrrolidine-2-carboxylic acid methyl ester (1)

To a suspension of Boc-L-tert-leucine (500 mg, 2.2 mmol) and HOBt (328 mg, 2.4 mmol) in CH₂Cl₂ (20 mL) cooled to 0° C. in an ice bath was added EDC (495 mg, 2.6 mmol). After stirring for 30 min, the reaction mixture was cooled down to 0° C. L-Hydroxyproline methyl ester hydrochloride (432 mg, 2.4 mmol) and NMM (0.6 mL, 5.4 mmol) were sequentially added. The reaction solution was allowed to warm up to room temperature and stirred for 3.5 h. The reaction mixture was diluted with additional CH₂Cl₂ (5 mL) and washed with water (8 mL). The organic layer was dried over Na₂SO₄ and evaporated under reduced pressure. The resulting oily residue was purified by silica gel column chromatography (solvent eluent gradient from 1:9 EtOAc/hexane to 9:1 EtOAc/hexane) to afford 1 (529 mg, 67% yield). MS m/z (rel intensity) 381 (M+23)⁺(4), 281 (19), 259 (19), 146 (100).

### 1,3-Dihydro-isoindole-2-carboxylic acid 1-(2-tert-butoxycarbonylamino-3,3-dimethyl-butyryl)-5-methoxycarbonyl-pyrrolidin-3-yl ester (2)

Compound 1 (279 mg, 0.8 mmol) was dissolved in CH₂Cl₂ (8 mL) and CDI (152 mg, 0.9 mmol) was added in one portion at room temperature. The reaction mixture was stirred for 25 h. Isoindoline (0.27 mL, 2.3 mmol) was then added portion-wise over 5 h. After 24 h of additional stirring, the reaction was diluted with CH₂Cl₂ (8 mL) and sequentially washed with precooled (0° C.) 1N HCl (8 mL) and brine (8 mL). The organic layer was dried over Na₂SO₄ and evaporated under reduced pressure. The resulting oily residue was purified by silica gel column chromatography (solvent eluent gradient from 3:7 EtOAc/hexane to 1:1 EtOAc/hexane) to afford 2 (257 mg, 66%). MS m/z (rel intensity) 526 (M+23)⁺(4), 404 (8), 291 (100).

### 1,3-Dihydro-isoindole-2-carboxylic acid 1-(2-tert-butoxycarbonylamino-3,3-dimethyl-butyryl)-5-carboxy-pyrrolidin-3-yl ester (3)

To a solution of compound 2 (257 mg, 0.5 mmol) in a 3:2 mixture of THF/water (5 mL) was added LiOH (21.5 mg, 0.5 mmol) in one portion. After 3 h, a second portion of LiOH (4 mg, 0.1 mmol) was added. The reaction mixture was stirred for an additional hour. The reaction was then cooled down to 0° C. and 1N HCl (0.6 mL, 0.6 mmol) was added dropwise over 2 minutes. The reaction was diluted with CH₂Cl₂ (10 mL) and washed with brine (5 mL). The organic layer was dried over Na₂SO₄ and evaporated under reduced pressure to yield an oily residue. A white solid precipitated from this oily residue upon standing overnight. This solid was washed with a 30% EtOAc/Hexanes mixture (2×5 mL) and used in the next reaction without further purification. MS m/z (rel intensity) 512 (M+23)⁺(4), 390 (5), 277 (100).

### 1,3-Dihydro-isoindole-2-carboxylic acid 1-(2-tert-butoxycarbonylamino-3,3-dimethyl-butyryl)-5-[1-(2,9,9-trimethyl-3,5-dioxa-4-bora-tricyclo[6.1.1.02,6]dec-4-yl)-propylcarbamoyl]-pyrrolidin-3-yl ester (4).

To a solution of compound 3 (50 mg, 0.1 μmol) in a 4:1 mixture of CH₂Cl₂/DMF (4 mL) cooled to 0° C. was added BOP (50 mg, 0.1 mmol) in one portion. The reaction was cooled down to −20° C. and a solution of ⁱPr₂EtN (18 uL, 0.1 mmol) in CH₂Cl₂ (0.1 mL) was added dropwise. The reaction was stirred for 15 minutes between −20° C. and −10° C. and then for 30 minutes at room temperature. It was cooled down to −20° C. and 1-(2,9,9-Trimethyl-3,5-dioxa-4-bora-tricyclo[6.1.1.0²,6]dec-4-yl)-propylamine hydrochloride (31 mg, 0.1 mmol) was added in one portion followed by dropwise addition of a solution of ⁱPr₂EtN (20 uL, 0.1 mmol) in CH₂Cl₂ (0.2 mL). The reaction was stirred between −20° C. and −10° C. for 45 minutes and then allowed to warm up to room temperature. It was stirred for two additional hours. The reaction was then cooled down to 0° C. and 5% citric acid (3 mL, aqueous solution) was added dropwise. The organic phase was separated and washed with 5% NaHCO₃ (5 mL, aqueous solution). The organic layer was dried over Na₂SO₄ and evaporated under reduced pressure. The oily residue was purified by silica gel column chromatography (solvent eluent gradient from 1:3 EtOAc/hexane to 19:1 EtOAc/hexane) to afford 4 (14 mg, 20%). MS m/z (rel intensity) 731 (M+23)⁺(8), 709 (M+1)(13), 609 (38), 457 (100).

It will be understood by those of skill in the art that other compounds of Formula I may be prepared by slight modification of the procedures set forth herein by using different reagents incorporating a W group, such as a boronic acid group, to prepare P1 moieties, different N-containing heterocycles for coupling to hydroxyl or amino proline derivatives to prepare P2 moieties and different protected amino acids, isocyanates, or the like, for preparing P3 moieties.

### Example 2

### HCV-NS3/4a Protease Assay

### Materials

HCV NS3/4a of genotype 1b, 5-FAM/QXL520 fluorescence resonance energy transfer (FRET) peptide, and buffer were purchased from Anaspec, San Jose. The sequence of this FRET peptide is derived from the cleavage site of NS4a/NS4b. IC_{50/90} calculations were performed by non-linear regression analysis using Prism software (GraphPad).

### Methods

Biochemical assay. Either 5 μL of DMSO or 5 μL of compound solution in DMSO at various concentrations is added to 45 μL of buffer containing 5 ng of NS3/4a per well in a 96 well plates for “enzyme only” and “compound testing” wells. “No enzyme” wells contain 45 μL of reaction buffer without the enzyme and 5 μL of DMSO. Plates are preincubed at room temperature for 1 hour. Protease reaction is initiated by addition of 50 μL of NS3/4a protease substrate solution to give a final substrate concentration of 2 μM. After shaking gently for 60 second and incubating at room temperature for 5 min, each well is measured for fluorescence intensity at Ex/Em=490 nm/520 nM every 5 minutes for 30 min. IC₅₀ and IC₉₀ values are calculated by non-linear regression analysis using Prism software (GraphPad). Selected compounds of the invention have been found to have activity in this assay.

### Example 3

### Synthesis of Compound 43

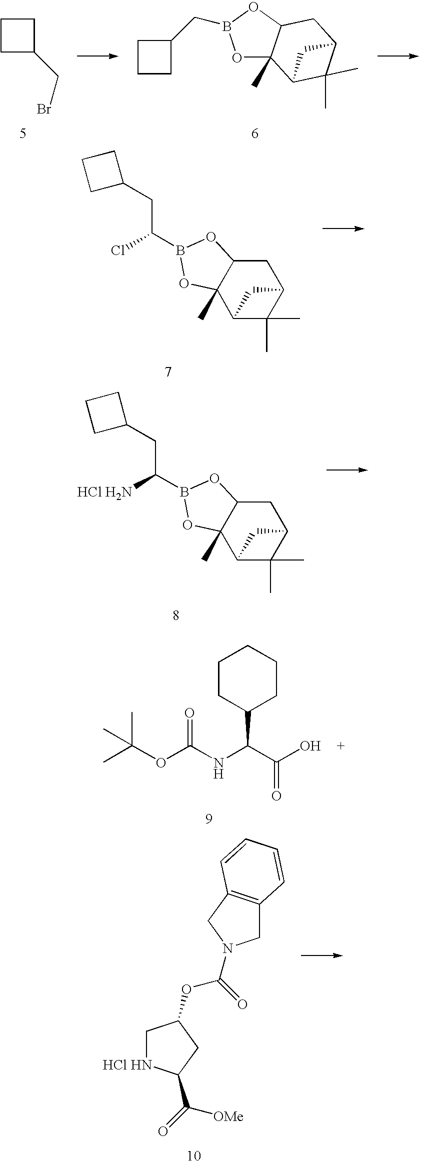

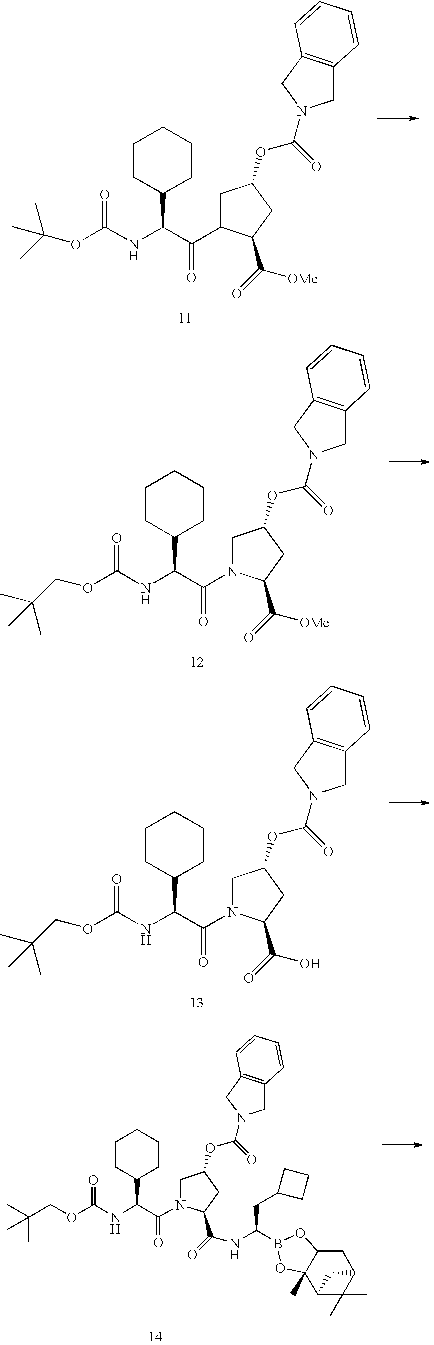

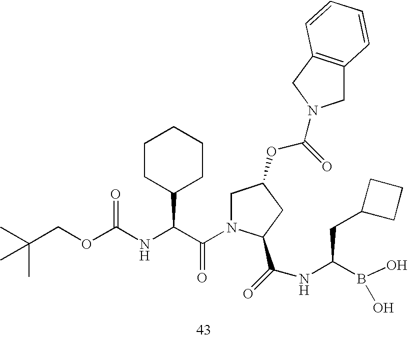

### Synthesis of 6; (+)Pinanediol 1-cyclobutyl-1-boronate

To a flame dried 2-necked round bottom flask equipped with a reflux condenser and magnetic stir bar charged with Et₂O (15 mL) and Mgo (200 mg, 8.37 mmol, 1.25 eq.). The suspension was stirred under a blanket of N₂ and an ethereal solution of 5 (1.0 g. 6.7 mmol) was slowly dripped into the flask until the suspension began to reflux. The remaining solution of 5 was added over a 10 min period and the reaction was heated at reflux for an additional 60 min. The resulting metallic grey suspension was cooled to rt, diluted with Et₂O (50 mL) and slowly dripped into a 0° C. solution of triisopropyl borate (1.43 mL, 6.7 mmol) in Et₂O (50 mL). The resulting cloudy solution was allowed to warm to rt and stirred for 4 hr followed by addition of 10% H₂SO₄ (50 mL). The biphasic solution was extracted with additional Et₂O (2×30 mL), washed with H₂O (50 mL), and brine (50 mL), dried over Na₂SO₄, and concentrated to approx half the original volume followed by addition of(+)-pinanediol (1.14 g, 6.7 mmol). The solution was stirred overnight, concentrated, and purified by flash column chromatography (silica gel, 1.5% EtOAc in hexanes) to give 6 (631 mg, 2.54 mmol, 38% yield) as a clear colorless oil. ¹H NMR (400 MHz, CDCl₃) δ 4.23 (dd, 1H); 2.48 (m, 1H); 2.18 (m, 1H); 2.10 (m, 2H), 2.04 (dd, 1H); 1.9 (m, 1H); 1.8 (m, 3H); 1.61 (m, 2H); 1.6 (s, 3H); 1.28 (s, 3H); 1.11 (d, 1H); 1.0 (d, 2H); 0.85 (s, 3H).

### Synthesis of 7; (+)Pinanediol 1-chloro-2-cyclobutyl-1-boronate

To a solution of dichloromethane (1.08 mL, 16.92 mmol) in THF cooled in a dry ice/acetone bath was carefully added n-BuLi (3.55 mL, 2.5 M solution in hexane, 8.87 mmol) by slowly dripping it down the sides of the reaction vessel. Upon completion the reaction was stirred at −78° C. for 1 hr. A solution of 6 (2.0 g, 8.06 mmol) dissolved in Et₂O (10 mL) was then slowly added to the reaction flask followed by ZnCl₂ (5.2 mL of a 1.0 M ethereal solution, 5.2 nmol, 0.65 eq). The reaction was allowed to warm slowly to room temperature over a 24 hr period. The amber colored solution was diluted with Et₂O (100 mL) followed by sat. NH₄Cl solution (100 mL), the organics were extracted and the water layer was washed with additional Et₂O (2×50 mL), organics were combined and washed with brine, dried over Na₂SO₄, and concentrated in vacuo to give a colorless oil that was purified by flash column chromatography (silica gel, 1.5% EtOAc in hexanes) to give a mix of 7 contaminated with starting material 2 in a 4:6 ratio respectively. MS m/z (rel intensity) 297 (M+1) (100).

### Synthesis of 8; (+)Pinanediol 1-amino-2-cyclobutyl-1-boronate hydrochloride

To a −78° C. solution of 7 (406 mg, 1.43 mmol) dissolved in THF (4 mL) under a balloon of dry N₂ was added LiHMDS (1.43 mL of a 1 M solution in THF) and the reaction was allowed to warm to room temperature overnight. The THF was removed and dichloromethane was added (˜30 mL) forming a white precipitate that was removed by filtration thru a plug of Celite. The filtrate was concentrated to near dryness, cooled to −78° C. followed by addition of HCl (4 N HCl in dioxane, 1.5 mL), warmed to room temperature and concentrated to give 8 as a brown sticky solid.

### Synthesis of 11; (1,3-Dihydro-isoindole-2-carboxylic acid 1-(2-tert-butoxycarbonylamino-2-cyclohexyl-acetyl)-5-methoxycarbonyl-pyrrolidin-3-yl ester)

To an ice cooled solution of 9 (480 mg, 1.8 mmol, 1.2 eq) in dichloromethane was added EDAC (450 mg, 1.5 eq) and HOBt (350 mg, 1.8 mmol, 1.2 eq). The solution stirred at 0° C. for 15 min then 10 (530 mg, 1.6 mmol) (see procedure for compound 10 below) and N-methyl morpholine (0.5 mL, 2.4 eq) were added sequentially. The reaction was warmed to room temperature overnight followed by addition of sat NaHCO₃ solution (20 mL). The organic layer was extracted with addition dichloromethane (2×20 mL), washed with 0.5 N HCl (40 mL) followed by brine (40 mL), dried over Na₂SO₄ and concentrated to give a tan solid. Further purification by flash column chromatography (silica gel, EtOAc/hexanes, 1:2) gave 11 (598 mg, 1.06 mmol, 67% yield) as a white solid. MS m/z (rel intensity) 529 (M+1) (8), 552 (M+22) (11), 291 (100).

### Synthesis of 12; (1,3-Dihydro-isoindole-2-carboxylic acid 1-[2-cyclohexyl-2-(2,2-dimethyl-propoxyarbonylamino)-acetyl]-5-methoxycarbonyl-pyrrolidin-3-yl ester)

To an ice cooled solution of 11 (471 mg, 0.83 mmol) in dichloromethane was added HCl (4 N in dioxane, 2.5 mL) and the reaction was warmed to room temperature overnight. The solvents were removed and the entire amount of crude product taken up in THF and neopentyl chloroformate (131 μL, 1.05 eq) was added. The reaction solution was cooled to −78° C. and NMM (340 mL, 1.67 mmol) was added. The reaction was warmed to room temperature, concentrated and purified by flash column chromatography (silica gel, EtOAc/hexanes, 1:2) to give 12 as a clear viscous oil (248 mg, 0.429 mmol, 53% yield).

### Synthesis of 13; (1,3-Dihydro-isoindole-2-carboxylic acid 5-carboxy-1-[2-cyclohexyl-2-(2,2-dimethyl-propoxycarbonylamino)-acetyl]-pyrrolidin-3-yl ester)

To an ice cooled solution of 12 (248 mg, 0.46 mmol) in a 30% H₂O in THF solution was added LiOH—H₂O (22 mg, 0.0548 mmol). The reaction was stirred at rt for 6 hrs, concentrated to near dryness followed by addition of 1 N HCl (550 μL, 0.55 nmol) in H₂O (10 mL), extracted with DCM (2×20 mL), washed with brine, dried over Na₂SO₄ and concentrated to give 13 (209 mg, 0.396 mmol, 87% yield) as a sticky white solid.

### Synthesis of 14; (1,3-Dihydro-isoindole-2-carboxylic acid 5-[1-(6,9,9-trimethyl-3,5-dioxa-4-bora-tricyclo[6.1.1.02,6]dec-4-yl)-2-cyclobutyl-ethylcarbamoyl)-1-[2-cyclohexyl-2-(2,2-dimethyl-propoxycarbonylamino)-acetyl]-pyrrolidin-3-yl ester)

To a solution of 13 (209 mg, 0.396 mmol) in dry THF was added isobutylchloroformate (53 mL, 0.395 mmol) was added N-methyl morpholine (86 mL, 0.414 mmol, 1.05 eq). Upon addition of the N-methyl morpholine a white precipitate immediately formed. The mixture was stirred for an additional 30 min followed by addition of 8 (125 mg, 0.396 mmol) and N-methyl morpholine (86 μL, 0.414 mmol, 1.05 eq). The reaction was warmed to room temperature overnight, concentrated to near dryness and then diluted with dichloromethane (20 mL) and sat NaHCO₃ solution (20 mL), extracted with additional dichloromethane (10 mL). The organics were combined and washed with 0.5 N HCl (20 mL), brine (20 mL), dried over Na₂SO₄, concentrated in vacuo, and purified by flash column chromatography (silica gel, 2% MeOH in dichloromethane) to give 14 (220 mg, 0.28 mmol, 70% yield) as a white solid. MS m/z (rel intensity) 789 (M+1) (100).

### Synthesis of 43; (1,3-Dihydro-isoindole-2-carboxylic acid 5-(1-boroxy-2-cyclobutyl-ethylcarbamoyl)-1-[2-cyclohexyl-2-(2,2-dimethyl-propoxycarbonylamino)-acetyl]-pyrrolidin-3-yl ester)

To a solution of 14 (140 mg, 0.177 mmol) in dichloromethane (2 mL) cooled in a dry ice/acetone bath was added BCl₃ (1.5 mL of a 1 M in dichloromethane). The reaction was allowed to warm to rt overnight followed by addition of dichloromethane (15 mL) and H₂O (15 mL). The organic layer was extracted with additional dichloromethane (2×10 mL), washed with brine, dried over Na₂SO₄ and purified by flash column chromatography (silica gel, eluent gradient from 5% MeOH in DCM to 15% MeOH) gave 43 as tan solid. MS m/z (rel intensity) 637 (M-H₂O) (100).

### Example 4

### Synthesis of Compound 10

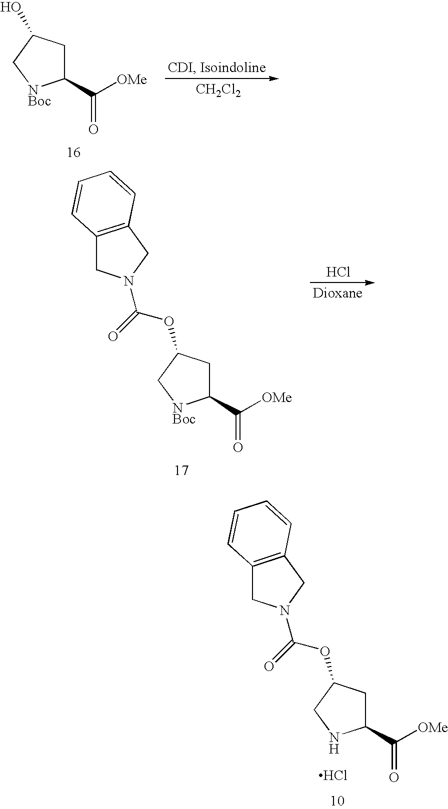

### Synthesis of 17; 4-(1,3-Dihydro-isoindole-2-carbonyloxy)-pyrrolidine-1,2-dicarboxylic acid 1-tert-butyl ester 2-methyl ester

Compound 16 (397 mg, 1.6 mmol) was dissolved in CH₂Cl₂ (10 mL) and CDI (315 mg, 1.9 mmol) was added in one portion at room temperature. The reaction mixture was stirred for 20 h. Isoindoline (0.55 ml, 4.8 mmol) was then added portion-wise over 8 h. After 20 h of additional stirring, the reaction was cooled down 0° C., diluted with CH₂Cl₂ (8 mL) and sequentially washed with aqueous 1N HCl (8 ml) and brine (8 ml). The organic layer was dried over Na₂SO₄ and evaporated under reduced pressure. The resulting oily residue was purified by silica gel column chromatography (solvent eluent gradient from 3:7 EtOAc/hexane to 6:4 EtOAc/hexane) to afford 17 (315 mg, 51%). MS m/z (rel intensity) 413 (M+23)⁺(6), 291 (23), 128 (100).

### Synthesis of 10; 1,3-Dihydro-isoindole-2-carboxylic acid 5-methoxycarbonyl-pyrrolidin-3-yl hydrochloride salt

Compound 17 (315 mg, 0.81 mmol) was dissolved in 4N HCl in dioxane (8 mL). The reaction was stirred at room temperature for 1.5 h. Solvents were removed under reduced pressure to yield 10 as a white solid which was used in the next reaction without further purification. MS m/z (rel intensity) 291 (M+1)⁺(4), 146 (17), 128 (100).

While the invention has been described and exemplified in sufficient detail for those skilled in this art to make and use it, various alternatives, modifications, and improvements will be apparent to those skilled in the art without departing from the spirit and scope of the claims.

What is claimed is:

1. A compound of Formula I: 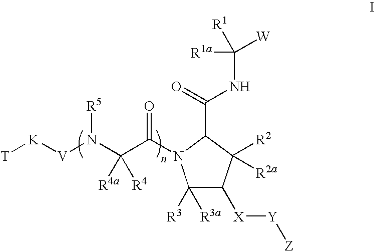
and stereoisomers, solvates, hydrates, tautomers, prodrugs, salts, pharmaceutically acceptable salts, and mixtures thereof, wherein:
n is 0 or 1;
W is 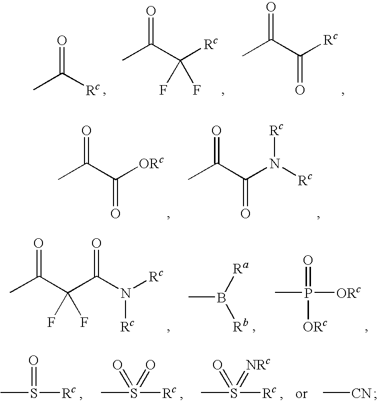
wherein
R^{a} and R^{b} are independently a hydroxyl or a group that can be converted to hydroxyl, or R^{a} and R^{b} together with the boron to which they are attached form a cyclic group which can be converted to a —B(OH)₂ group;
R^{c} at each occurrence is independently H, substituted or unsubstituted alkyl, alkenyl, aryl, aralkyl, aralkenyl, cycloalkyl, cycloalkylalkyl, cycloalkenyt, cycloalkenylalkyl, heterocyclyl, heterocyclylalkyl, heterocyclylalkenyl, heteroaryl, or heteroarylalkyl; or two R^{c} groups bound to a nitrogen atom can together with the nitrogen atom to which they are bound form a 5-7 membered monocyclic heterocyclic ring system; wherein any carbon atom of R^{c} can be substituted with J;
R¹ and R^{1a} are independently H or a substituted or unsubstituted alkyl, alkenyl, alkynyl, cycloalkyl, cycloalkylalkyl, aryl, or aralkyl group; or R¹ and R^{1a} together with a carbon atom to which they are attached form together with the carbon atom a 3-7 membered substituted or unsubstituted carbocycle; wherein each of R¹ or R^{1a} can be substituted with 0-3 J;
R², R^{2a}, R³ and R^{3a} are independently H or a substituted or unsubstituted alkyl, alkenyl, aryl, aralkyl, aralkenyl, cycloalkyl, cycloalkylalkyl, cycloalkenyl, cycloalkenylalkyl, heterocyclyl, heterocyclylalkyl, heteroaryl, or heteroarylalkyl group, wherein any carbon atom can be substituted with J;
R⁴ and R^{4a} are independently hydrogen, (C₁-C₁₂)-aliphatic, (C₃-C₁₀)-cycloalkyl or cycloalkenyl, [(C₃-C₁₀)cycloalkyl or cycloalkenyl]-(C₁-C₁₂)-aliphatic, (C₆-C₁₀)-aryl, (C₆-C₁₀)-aryl-(C₁-C₁₂)-aliphatic, (C₃-C₁₀)-heterocyclyl, (C₃-C₁₀)-heterocyclyl-(C₁-C₁₂)-aliphatic, (C₅-C₁₀)-heteroaryl, or (C₅-C₁₀)-heteroaryl-(C₁-C₁₂)-aliphatic;
wherein each (C₁-C₁₂)-aliphatic, (C₃-C₁₀)-cycloalkyl or cycloalkenyl, [(C₃-C₁₀)cycloalkyl or cycloalkenyl]-(C₁-C₁₂)-aliphatic, (C₆-C₁₀)-aryl, (C₆-C₁₀)-aryl-(C₁-C₁₂)-aliphatic, (C₃-C₁₀)-heterocyclyl, (C₃-C₁₀)-heterocyclyl-(C₁-C₁₂)-aliphatic, (C₅-C₁₀)-heteroaryl, or (C₅-C₁₀)-heteroaryl-(C₁-C₁₂)-aliphatic of R⁴ or R^{4a} is independently substituted with 0-3 substituents independently selected from J;
wherein up to 3 carbon atoms in each of R⁴ or R^{4a} may be replaced by a heteroatom selected from N, NH, O, S, SO, or SO₂ in a chemically stable arrangement; or wherein R⁴ and R^{4a} together with a carbon atom to which they are bound form a 3- to 8-membered ring having up to 3 heteroatoms selected from N, NH, O, S, SO, or SO₂, wherein the ring system is substituted with 0-2 substituents selected independently from J;
R⁵ is hydrogen, alkyl, alkenyl, cycloalkyl, cycloalkylalkyl, cycloalkenyl, cycloalkyenylalkyl, aryl, aralkyl, heterocyclyl, heterocyclylalkyl, heteroaryl or heteroarylalkyl; wherein the alkyl, alkenyl, cycloalkyl, cycloalkylalkyl, cycloalkenyl, cycloalkenylalkyl, aryl, aralkyl, heterocyclyl, heterocyclylalkyl, heteroaryl or heteroarylalkyl is substituted with 0-3 J groups;
X is a bond, C(H)R⁷, O, S, or N(R⁷);
Y is a bond, C(H)R⁷, C(O), C(O)C(O), S(O), S(O)₂, or S(O)(NR⁷);
wherein R⁷ is hydrogen, alkyl, cycloalkyl, cycloalkylalkyl, aryl, aralkyl, heterocyclyl, heterocyclylalkyl, heteroaryl, heteroarylalkyl, aralkanoyl, heteroaralkanoyl, C(O)R⁸, C(O)OR⁸, SO₂R⁸, or carboxamido, and the alkyl, cycloalkyl, cycloalkylalkyl, aryl, aralkyl, heterocyclyl, heterocyclylalkyl, heteroaryl, heteroarylalkyl, aralkanoyl, or heteroaralkanoyl is substituted with 0-3 J groups, or R⁷ and Z, together with the atoms to which they are bound, form a mono- or bicyclic ring system substituted with 0-3 J groups;
wherein R⁸ is alkyl, aryl, aralkyl, heterocyclyl, heterocyclylalkyl, heteroaryl or heteroarylalkyl, any of which is substituted with 0-3 J groups;
Z is alkyl, aryl, aralkyl, cycloalkyl, cycloalkylalkyl, cycloalkenyl, cycloalkenylalkyl, heterocyclyl, heterocyclylalkyl, heteroaryl, heteroarylalkyl, OR⁹, or N(R⁹)₂, wherein any carbon atom can be substituted with J;
wherein each R⁹ is independently hydrogen, alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroarylalkyl, the alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroarylalkyl being substituted with 0-3 J groups; or two R⁹ groups which are bound to a nitrogen atom form together with the nitrogen atom a 3- to 8-membered mono-, or an 8- to 20-membered bi- or tricyclic heterocyclic ring system substituted with 0-3 J groups;
J is halogen, OR′, OC(O)N(R′)₂, NO₂, CN, CF₃, OCF₃, R′, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′.)₂, N(R′)SO₂R′, (CH₂)₀₋₂N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, C(═NOR′)R′, OP(O)(OR′)₂, P(O)(R′)₂, P(O)(OR′)₂, or P(O)(H)(OR′); or two J groups taken together are O, S, C(O), S(O), S(O)₂, methylenedioxy, or ethylenedioxy;
wherein each R′ is independently selected from hydrogen, (C₁-C₁₂)-aliphatic, (C₃-C₁₀)-cycloalkyl or cycloalkenyl, [(C₃-C₁₀)cycloalkyl or cycloalkenyl]-(C₁-C₁₂)-aliphatic, (C₆-C₁₀)-aryl, (C₆-C₁₀)-aryl-(C₁-C₁₂)-aliphatic, (C₃-C₁₀)-heterocyclyl, (C₃-C₁₀)-heterocyclyl-(C₁-C₁₂)-aliphatic, (C₅-C₁₀)-heteroaryl, or (C₅-C₁₀)-heteroaryl-(C₁-C₁₂)-aliphatic, or wherein two R′ groups together with a nitrogen atom to which they are bound form together with the nitrogen atom a 3- to 8-membered mono-, or an 8- to 20-membered bi- or tricyclic heterocyclic ring system; wherein, in the bi- and tricyclic ringsystems, each ring is linearly fused, bridged, or spirocyclic; wherein each ring is either aromatic or nonaromatic; wherein each heteroatom in the heterocyclic ring system is selected from the group consisting of N, NH, O, S, SO and SO₂; wherein any R′ other than hydrogen is substituted with 0-3 substituents selected independently from J;
V is a bond, CH₂, C(R¹⁰)₂, C(O), S(O), or S(O)₂;
K is a bond, —O—, —S—, —C(O)—, —S(O)—, S(O)₂—, —S(O)(NR¹⁰)—, or —N(R¹⁰)—;
wherein R¹⁰ is hydrogen or C₁₋₅ alkyl;
T is R¹¹, R¹¹-alkyl-, R¹¹-alkenyl-, R¹¹-alkynyl-, R¹¹O—, —N(R¹¹)₂, —C(O) R¹¹, or
—C(═NOalkyl) R¹¹;
wherein R¹¹ is independently hydrogen, alkyl, aryl, aralkyl, alkoxy, cycloalkyl, cycloalkylalkyl, cycloalkenyl, cycloalkenylalkyl, heterocyclyl, heterocyclylalkyl, heterocyclylalkenyl, or heteroaryl, and each alkyl, aryl, aralkyl, alkoxy, cycloalkyl, cycloalkylalkyl, cycloalkenyl, cycloalkenylalkyl, heterocyclyl, heterocyclylalkyl, heterocyclylalkenyl, or heteroaryl is substituted with 0-3 J groups; or a first R¹¹ and a second R¹¹ bonded to a nitrogen atom together with the nitrogen atom to which they are bound form a mono- or bicyclic ring system substituted with 0-3 J groups.

2. The compound of claim 1, wherein Z is unsubstituted or substituted heterocyclyl.

3. The compound of claim 1, wherein Z is N(R⁹)₂ and wherein the two R⁹ groups, together with a nitrogen atom to which they are bound form together with the nitrogen atom a 3- to 8-membered monocyclic heterocyclic ring system or an 8- to 20-membered bicyclic heterocyclic ring system wherein the heterocyclic ring system is substituted with 0-3 J groups.

4. The compound of claim 1, wherein Z is 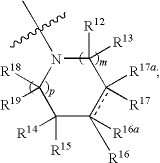
wherein
the bond including a dashed line can be a single bond or a double bond;
m is 0 or 1;
p is 0 or 1;
R¹², R¹³, R¹⁸, and R¹⁹ are independently hydrogen or a substituted or unsubstituted alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group; or R¹² and R¹³, or R¹⁸ and R¹⁹, together with a carbon atom to which they are attached form a C₃₋₆ cycloalkyl group;
R¹⁴ and R¹⁵ are independently hydrogen, fluorine, or a substituted or unsubstituted alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group, or R¹⁴ and R¹⁵, together with a carbon atom to which they are attached form a C₃₋₆ cycloalkyl group;
wherein any R¹², R¹³, R¹⁴, R¹⁵, R¹⁸, or R¹⁹ alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group is substituted with 0-3 J groups; and wherein any C₃₋₆ cycloalkyl group formed by R¹² and R¹³, or R¹⁴ and R¹⁵, or R¹⁸ and R¹⁹, together with a carbon atom to which they are bonded, can comprise 1 or 2 heteroatoms selected from a group consisting of O, NH, NR′, S, SO, and SO₂;
R¹⁶, R^{16a}, R¹⁷ and R^{17a} are independently hydrogen or a substituted or unsubstituted alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group; or R¹⁶ and R¹⁷ together with the atoms to which they are attached form a fused substituted or unsubstituted aryl or heteroaryl group; or when the bond including the dashed line is a double bond, R^{16a} and R^{17a} are absent;
wherein the wavy line signifies a point of attachment.

5. The compound of claim 4, wherein R¹², R¹³, R¹⁴, R¹⁵, R¹⁶, R¹⁷, R¹⁸, and R¹⁹, and R^{16a} and R^{17a}, if present, are hydrogen.

6. The compound of claim 1, wherein Z is 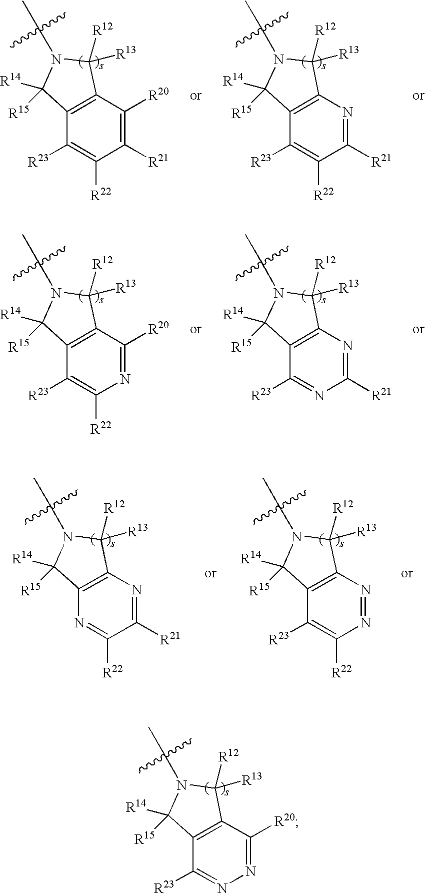
wherein s is 1 or 2;
R¹², R¹³, R¹⁴, and R¹⁵ are at each occurrence independently hydrogen, fluorine, or an alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group; or R¹² and R¹³, or R¹⁴ and R¹⁵, together with a carbon atom to which they are bonded form a C₃₋₆ cycloalkyl group;
wherein any R¹², R¹³, R¹⁴, or R¹⁵ alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group is substituted with 0-3 J groups; and wherein any C₃₋₆ cycloalkyl group formed by R¹² and R¹³, or R¹⁴ and R¹⁵, together with a carbon atom to which they are bonded, can comprise 1 or 2 heteroatoms selected from a group consisting of O, NH, NR′, S, SO, and SO₂;
R²⁰, R²¹, R²², R²³ are a substituted or unsubstituted alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group, or are independently H, F, Cl, Br, I, NO₂, CN, CF₃, OR²⁴, O—(CH₂)ᵣ—NR²⁵R²⁶, O—(CH₂)ᵣ—OC(O)NR²⁵R²⁶, O—(CH₂)ᵣ—NR²⁵C(O)OR²⁶, (CH₂)—OR²⁴, OCF₃, NR²⁵R²⁶, (CH₂)ᵣ—NR²⁵R²⁶, SR²⁴, (CH₂)ᵣ—SR²⁴, C(O)R²⁴, C(O)OR²⁴, NR²⁷C(O)R²⁴, C(O)NR²⁵R²⁶, NR²⁷C(O)NR²⁵R²⁶, OC(O)NR²⁵R²⁶, NR²⁷C(O)OR²⁴, NR²⁷SO₂R²⁴, SO₂ NR²⁵R²⁶, wherein r is 1, 2, 3, 4, 5, or 6; and
each R²⁴, R²⁵, R²⁶, and R²⁷ is independently hydrogen or an alkyl, cycloalkyl, cycloalkenyl, cycloalkylalkyl, cycloalkenylalkyl, cycloalkylalkenyl, aryl, aralkyl, arylalkenyl, heterocyclyl, heterocyclylalkyl, heterocyclylalkenyl, heteroaryl, heteroarylalkyl, or heteroarylalkenyl group, wherein any R²⁴, R²⁵, R²⁶, or R²⁷ group except hydrogen is substituted with 0-3 J groups; or R²⁵ and R²⁶ together with a nitrogen atom to which they are attached form together with the nitrogen atom a 3-7 membered heterocyclic ring which is substituted with 0-3 J groups;
wherein the wavy line signifies a point of attachment.

7. The compound of claim 6 wherein R¹², R¹³, R¹⁴R¹⁵, and R²⁰, R²¹, R²² and R²³, when present, are hydrogen.

8. The compound of claim 1, wherein R¹ is alkyl, cycloalkyl, or cycloalkylalkyl, and R^{1a} is H; or wherein R¹ and R^{1a} together with a carbon atom to which they are attached form together with the carbon atom a 3-, 4-, or 5-membered cycloalkyl, wherein any 1 or 2 carbon atoms of R¹, or of R¹ and R^{1a} combined in the cycloalkyl, may be replaced by 1 or 2 heteroatoms respectively, selected from the group consisting of O, NH, NR′, S, SO or SO₂; wherein any carbon atom of R¹ or of R¹ and R^{1a} combined in the cycloalkyl may be unsubstituted or substituted with a J group.

9. The compound of claim 8, wherein R¹ is ethyl, n-propyl, isopropyl, butyl, isobutyl, cyclopropylmethyl, or cyclobutylmethyl.

10. The compound of claim 1, wherein R⁴ is alkyl, cycloalkyl, or cycloalkylalkyl.

11. The compound of claim 1, wherein R⁴ is isopropyl, t-butyl, sec-butyl, cyclopropyl, cyclohexyl, 4-hydroxycyclohexyl, or 4-(C₁₋₆)alkoxycyclohexyl, and R^{4a} is H.

12. The compound of claim 1, wherein V is C(O).

13. The compound of claim 1, wherein K is O, NH, or N(CH₃).

14. The compound of claim 1, where T is alkyl, aralkyl, or heteroarylalkyl, wherein any alkyl, aralkyl, or heteroarylalkyl is substituted with 0-3 J groups.

15. The compound of claim 1, wherein T is methyl, ethyl, propyl, isopropyl, sec-butyl, isobutyl, t-butyl, hydroxy-t-butyl, neopentyl, 2,2-dimethylbutan-3-yl, 2-methylbutan-3-yl, benzyl, 2-fluoroethyl, 2-methoxyethyl, 2-(diethylamino)ethyl, 3-(dimethylamino)propyl, thiazol-5-ylmethyl, tetrahydrofuran-2-ylmethyl, 1-(N-methyl-methansulfonamido)-3,3,-dimethylbutan-2-yl, 1-(N-methyl-methansulfonamido)-3-methylbutan-2-yl, 1-(N-methyl-methansulfonamido)butan-2-yl, 1-(N-methyl-methansulfonamido)-3-methylpentan-2-yl, 1-(N-methyl-cyclopropansulfonamido)-3,3,-dimethylbutan-2-yl, 2-(2-pyridyl)-2,2-difluoroethyl, hexahydropyran-4-ylmethyl, 1-(t-butylsulfonylmethyl)cyclohex-1-yl, 2-(N-methyl-methansulfonamido)ethyl, 1-(N-methyl-methansulfonamido)prop-2-yl, or 2-(N-methyl-methansulfonamido)-1-cyclohexyl-ethyl.

16. The compound of claim 1, wherein X is O or N(R⁷).

17. The compound of claim 16, wherein R⁷ is H.

18. The compound of claim 1, wherein Y is C(O).

19. The compound of claim 1, wherein R⁵ is hydrogen or methyl.

20. The compound of claim 1, wherein W is —B(OH)₂.

21. The compound of claim 1, wherein R², R^{2a}, R³ and R^{3a} are hydrogen.

22. The compound of claim 1, wherein the compound of Formula I is:

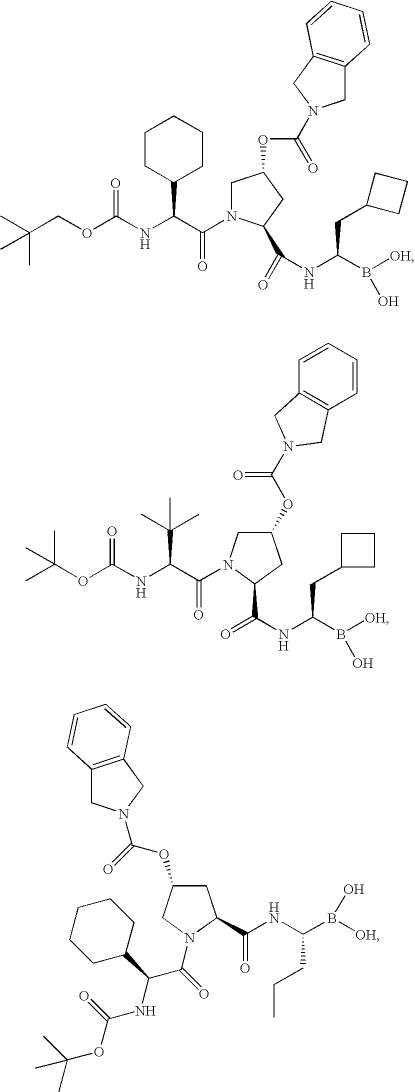

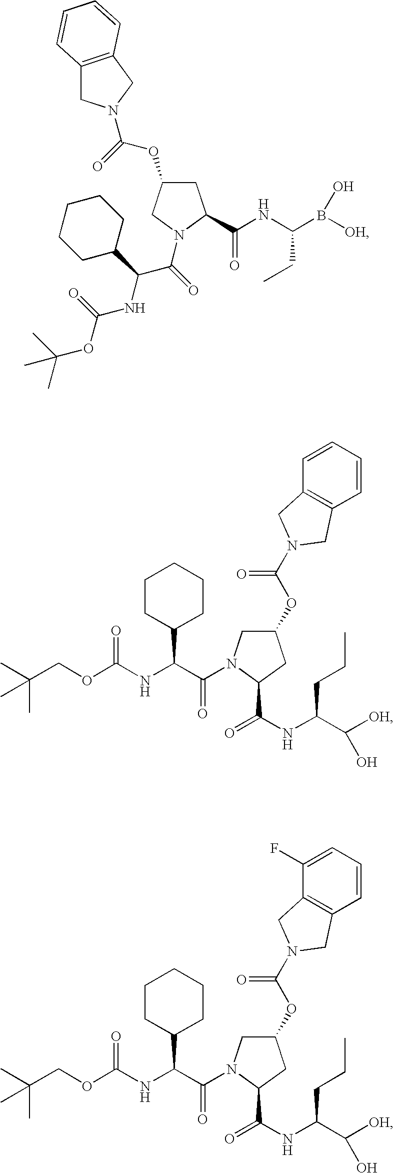

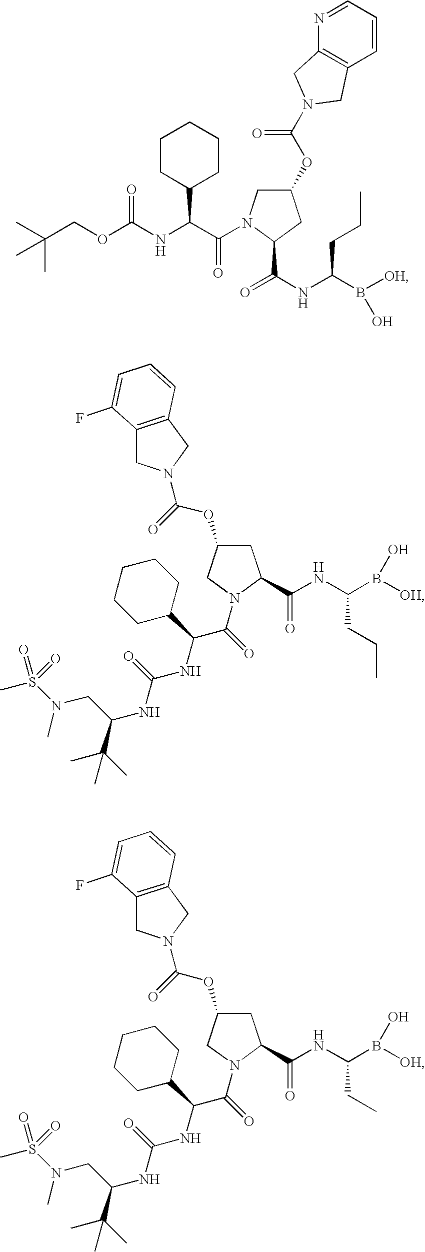

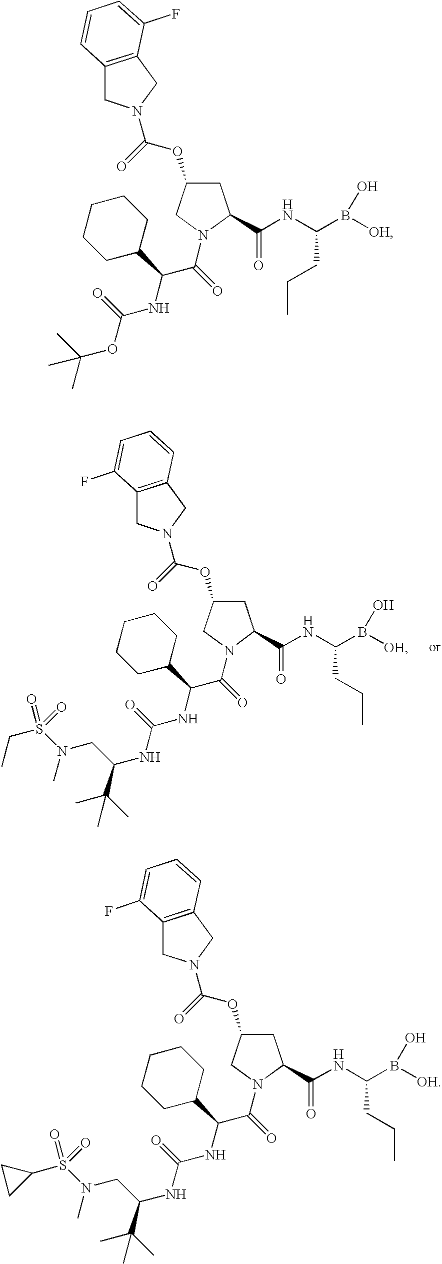

23. A method of preparation of a compound of Formula I of claim 1, comprising contacting a compound of Formula II: 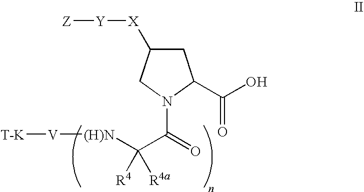
with a compound of Formula III: 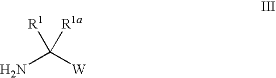
under conditions adapted to bring about formation of an amide bond between a carboxyl group of the compound of Formula II and an amino group of the compound of Formula III.

24. The method of claim 23, comprising use of reagents benzotriazol-1-yl-oxy-tris-(dimethylamino)phosphonium hexa-fluorophosphate (BOP) and diisopropylethylamine (DIEA) to bring about the formation of the amide bond.

25. The method of claim 23, wherein the conditions comprise temperatures of <0° C.

26. The method of claim 23, comprising use of solvents dichloromethane or N,N-dimethylformamide, or both.

27. A pharmaceutical composition comprising a compound of claim 1 and a suitable excipient.

28. A pharmaceutical combination comprising a compound of claim 1 in a therapeutically effective dose and an additional medicament or a plurality of additional medicaments in therapeutically effective amounts.

29. A pharmaceutical composition comprising the combination of claim 28 and a suitable excipient.

30. The use of a compound of claim 1 for preparation of a medicament for the treatment of hepatitis C.

31. The use of claim 30 further comprising use of an additional medicament or a plurality of additional medicaments for preparation of a medicament for the treatment of hepatitis C.

32. A method of treatment of a malcondition in a patient in need thereof, wherein inhibition of a hepatitis C viral protease is medically indicated, comprising administering to the patient a compound of claim 1 in a therapeutically effective amount.

33. A method of treatment of a malcondition in a patient, the malcondition comprising a hepatitis C viral infection, the method comprising administering to the patient a compound of claim 1 in a therapeutically effective amount.

34. The method of claims 32 or 33 further comprising administering to the patient an additional medicament or a plurality of additional medicaments in therapeutically effective amounts.

35. The method of claim 34 wherein the additional medicament or plurality of additional medicaments comprises an anti-viral compound.

36. The method of claim 35 wherein the additional medicament or plurality of additional medicaments comprises another compound of claim 1, another HCV protease inhibitor, interferon alfa-2b, peginteferon alfa-2b, recombinant interferon alfa-2a, peginterferon alfa-2a, inteferon-alpha 2B+Ribavirin, interferon alpha-n1, nucleoside analogues, IRES inhibitors, NS5b inhibitors, E1 inhibitors, E2 inhibitors, IMPDH inhibitors, NS5 polymerase inhibitors, or NTPase/helicase inhibitors.

37. The method of claim 34 wherein the additional medicament or plurality of additional medicaments comprises an anti-proliferative agent.

38. The method of claim 37, wherein the anti-proliferative agent comprises 5-fluorouracil, daunomycin, mitomycin, bleomycin, dexamethasone, methotrexate, cytarabine, or mercaptopurine.

39. The method of claim 34, wherein the additional medicament or plurality of additional medicaments comprises an immune modulator.

40. The method of claim 39, wherein the immune modulator comprises a steroid, a non-steroidal anti-inflammatory, a COX2 inhibitor, an anti-TNF compound, an anti-IL1 compound, an interferon, methotrexate, leflunomide, cyclosporin, FK506, or a combination of any two or more thereof.

41. The method of claim 40 wherein the steroid is prednisone, prednisolone, or dexamethasone.

42. The method of claim 40 wherein the non-steroidal anti-inflammatory is ibuprofen, naproxen, diclofenac, or indomethacin.

43. The method of claim 40, wherein the COX2 inhibitor is rofecoxib or celecoxib.

44. The method of claim 40, wherein the anti-TNF compound is enbrel, infliximab, or adaumimab.

45. The method of claim 40, wherein the anti-ILl compound is anakinra.

46. The method of claim 40, wherein the interferon is interferon alfa-2b, peginteferon alfa-2b, recombinant interferon alfa-2a, peginterferon alfa-2a, or interferon alpha-n1.
